(19) United States
(12) Patent Application Publication
(10) Pub. No.: US 2023/0217981 A1
(43) Pub. Date: Jul. 13, 2023

(54) High Purity Protein Preparation from Plant Material and Products Thereof

(71) Applicant: Plantible Foods Inc., San Marcos, CA (US)

(72) Inventors: Tony MARTENS, San Marcos, CA (US); Cesar FLORES, El Centro, CA (US); Eduardo REYNOSO MORENO, San Diego, CA (US); Roland VAN DER MOLEN, Carlsbad, CA (US)

(21) Appl. No.: 18/121,226

(22) Filed: Mar. 14, 2023

Related U.S. Application Data
(63) Continuation of application No. 17/625,468, filed on Jan. 7, 2022.
(60) Provisional application No. 62/872,917, filed on Jul. 11, 2019.

(51) Int. Cl. A23L 33/185; C07K 1/34; A23J 1/00; A23J 3/34; C07K 1/14; C07K 1/32; C12N 9/88

(52) U.S. Cl. CPC A23L 33/185; C07K 1/34; A23J 1/006; A23J 3/346; C07K 1/145; C07K 1/32; C12N 9/88; C12Y 401/01039; A23V 2002/00

(57) ABSTRACT

Processes for preparing and purifying protein from plant material, and compositions and uses comprising the same, are provided.

### CROSS-REFERENCE TO RELATED APPLICATIONS

This application is a continuation application of U.S. application Ser. No. 17/625,468, filed Jan. 7, 2022, which is a national phase application of PCT/US2020/041525, filed Jul. 10, 2020, which claims the benefit of U.S. Provisional Application No. 62/872,917, filed on Jul. 11, 2019, the contents of which are incorporated by reference in their entirety.

### FIELD OF THE DISCLOSURE

Disclosed are processes for making, extracting, and purifying a protein preparation from plant material, and the products made from such processes.

### BACKGROUND

The production of meat for human consumption has a large negative environmental impact. The industry is known to be one of the largest emitters of greenhouse gases and a leading cause of water pollution and loss of biodiversity. Steinfeld et al., Livestock's Long Shadow: Environmental Issues and Options, (2006) Food and Agriculture Organization of the United Nations; Machovina et al., Science of the Total Environment, (2016) 536: 419-431; Godfray et al., Science, (2018) 361(6399):eaam5324. As the world consumption of meat increases due to population, wealth, and lifestyle changes there is a growing need for alternatives to meat that are environmentally friendly. Erb et al., Nat. Comms., (2016) 7:11382.

Replacement of meat in the diet requires plant-based high-protein substitutes that can be sustainably produced on a large scale. The protein ribulose-1,5-bisphosphate carboxylase/oxygenase (“RuBisCo”) comprises up to 50% of the total protein in plants. It is the enzyme involved in the first major step of carbon fixation, a process by which atmospheric carbon dioxide is converted by plants and other photosynthetic organisms to energy-rich molecules such as glucose. Due to its abundance in plants, it serves as an alternative source of protein for food production. Purified RuBisCo is typically a tasteless, odorless, white powder.

The duckweed (subfamily Lemnoideae, genus Lemna) is among the smallest flowering plants in the world. Despite its diminutive size, it has the ability to grow quickly, doubling its biomass in about 16 to 48 hours depending on the conditions. Mestameyer et al., Spirodela punctata Aquatic Botany, (1984) 19:157-70. Lemna has a high protein content (about 30-35% of its dry mass being protein) and thus has been used as in animal feedstock. All these properties make Lemna an attractive candidate for large scale production of plant-based protein for food.

The ability of proteins to form emulsions, gels and stable foams are also important in the production of a variety of foods, forming the basis for texture in the food products. For example, foams with a uniform distribution of small air bubbles impart body, smoothness and lightness to the food. The ability of a protein preparation to form a foam is related to its purity, and a purity of at least about 80% may be needed to form a stable foam. Similarly, gels made from proteins give rise to foods of different rheological properties and appearances. The gelling capacity of a protein may be measured by the amount of protein needed to form a gel. Thus, protein preparations with high purity, foaming capacity, foam stability and gelling capacity are desired for use in food products.

There is currently a need for economical processes to purify and extract protein from plants, e.g., from Lemnoideae or Lemna. Disclosed herein are processes that can produce purified protein preparations that have the flexibility to be formulated into most food products.

### SUMMARY

The present disclosure provides a process for making a purified protein preparation from a plant material, comprising:

a) providing the plant material in a buffer solution comprising a reducing agent;

b) lysing the plant material;

c) separating the lysed plant material into a solid phase and a liquid phase, wherein the liquid phase contains soluble protein and chlorophyll;

d) coagulating the chlorophyll in the liquid phase by heating it to a first set temperature in no more than about 30 min, then cooling it to a second set temperature in no more than about 30 min, wherein the cooling is initiated when the liquid phase reaches the first set temperature.

e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;

f) separating the flocculated mixture of e) into a solid phase and a liquid phase; and

g) filtering the liquid phase of f) to yield a filtrate containing a purified protein.

The present disclosure also provides a process for making a purified protein preparation from a plant material, comprising:

a) providing the plant material in a buffer solution comprising a reducing agent;

b) lysing the plant material;

c) separating the lysed plant material into a solid phase and a liquid phase, wherein the liquid phase contains soluble protein and chlorophyll;

d) coagulating the chlorophyll in the liquid phase by adding one or more salts;

e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;

f) separating the flocculated mixture of e) into a solid phase and a liquid phase; and

g) filtering the liquid phase of f) to yield a filtrate containing a purified protein.

The present disclosure also provides a process for making a purified protein preparation from a plant material, comprising:

a) providing the plant material in a buffer solution comprising a reducing agent;

b) lysing the plant material;

c) separating the lysed plant material into a solid phase and a liquid phase, wherein the liquid phase contains soluble protein and chlorophyll;

d) coagulating the chlorophyll in the liquid phase by adding one or more salts;

e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;

f) separating the flocculated mixture of e) into a solid phase and a liquid phase; and

g) filtering the liquid phase of f) to yield a filtrate containing a purified protein.

The present disclosure also provides a process for making a purified protein preparation from a plant material, comprising:

a) providing the plant material in a buffer solution comprising a reducing agent;
b) lysing the plant material;
c) separating the lysed plant material into a solid phase and a liquid phase, wherein the liquid phase contains soluble protein and chlorophyll;
d) coagulating the chlorophyll in the liquid phase using a polymer-based coagulant;
e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) separating the flocculated mixture of e) into a solid phase and a liquid phase; and
g) filtering the liquid phase of f) to yield a filtrate containing a purified protein.

The present disclosure also provides a process for making a purified protein preparation from a plant material, comprising:

a) providing the plant material in a buffer solution comprising a reducing agent;
b) lysing the plant material;
c) separating the lysed plant material into a solid phase and a liquid phase, wherein the liquid phase contains soluble protein and chlorophyll;
d) coagulating the chlorophyll in the liquid phase by electrocoagulation;
e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) separating the flocculated mixture of e) into a solid phase and a liquid phase; and
g) filtering the liquid phase of f) to yield a filtrate containing a purified protein.

In some aspects, the present disclosure relates to the following embodiments:

1. A process for making a purified protein preparation from a plant material, comprising:
a) providing the plant material in a buffer solution comprising a reducing agent;
b) lysing the plant material;
c) separating the lysed plant material into a solid phase and a liquid phase, wherein the liquid phase contains soluble protein and chlorophyll;
d) coagulating the chlorophyll in the liquid phase by heating it to a first set temperature in no more than about 30 min, then cooling it to a second set temperature in no more than about 30 min, wherein the cooling is initiated when the liquid phase reaches the first set temperature;
e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) separating the flocculated mixture of e) into a solid phase and a liquid phase; and
g) filtering the liquid phase of f) to yield a filtrate containing a purified protein.
2. A process for making a purified protein preparation from a plant material, comprising:
a) providing the plant material in a buffer solution comprising a reducing agent;
b) lysing the plant material;
c) separating the lysed plant material into a solid phase and a liquid phase, wherein the liquid phase contains soluble protein and chlorophyll;
d) coagulating the chlorophyll in the liquid phase by adding one or more salts;
e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) separating the flocculated mixture of e) into a solid phase and a liquid phase; and
g) filtering the liquid phase of f) to yield a filtrate containing a purified protein.
3. A process for making a purified protein preparation from a plant material, comprising:
a) providing the plant material in a buffer solution comprising a reducing agent;
b) lysing the plant material;
c) separating the lysed plant material into a solid phase and a liquid phase, wherein the liquid phase contains soluble protein and chlorophyll;
d) coagulating the chlorophyll in the liquid phase using a polymer-based coagulant;
e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) separating the flocculated mixture of e) into a solid phase and a liquid phase; and
g) filtering the liquid phase of f) to yield a filtrate containing a purified protein.
4. A process for making a purified protein preparation from a plant material, comprising:
a) providing the plant material in a buffer solution comprising a reducing agent;
b) lysing the plant material;
c) separating the lysed plant material into a solid phase and a liquid phase, wherein the liquid phase contains soluble protein and chlorophyll;
d) coagulating the chlorophyll in the liquid phase by electrocoagulation;
e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) separating the flocculated mixture of e) into a solid phase and a liquid phase; and
g) filtering the liquid phase of f) to yield a filtrate containing a purified protein.
5. The process of any one of embodiments 1-4, wherein the plant material is washed before a).
6. The process of any one of embodiments 1-4, wherein the reducing agent is 2-mercaptoethanol (BME), 2-mercaptoethylamine-HCL, sodium metabisulfite, cysteine hydrochloride, dithiothreitol (DTT), glutathione, cysteine, tris(2-carboxyethyl)phosphine (TCEP), ferrous ion, nascent hydrogen, sodium amalgam, oxalic acid, formic acid, magnesium, manganese, phosphorous acid, potassium, or sodium.
7. The process of any one of embodiments 1-4, wherein the reducing agent is a sulfite.
8. The process of embodiment 7, wherein the sulfite is sodium sulfite, magnesium sulfite, or sodium metabisulfite.
9. The process of embodiment 7, wherein the sulfite is sodium bisulfate.
10. The process of any one of embodiments 1-4, wherein the solution in a) contains one or more buffering agents.
11. The process of any one of embodiments 1-4, wherein the solution in a) contains a one or more chelating agents.
12. The process of any one of embodiments 1-4, wherein the solution in a) contains one or more protease inhibitors.
13. The process of any one of embodiments 1-4, wherein the solution in a) contains one or more buffering agents, one or more chelating agents, and/or one or more protease inhibitors.
14. The process of any one of embodiments 1-4, wherein the pH of the solution in a) is about pH 5.0 to about pH 9.0.
15. The process of embodiment 14, wherein the pH of the solution is about pH 6.0 to about pH 7.6.
16. The process of embodiment 15, wherein the pH of the solution is about 6.8.
17. The process of any one of embodiments 1-4, wherein the plant material and solution of a) is at a ratio of about 6:1.
18. The process of any one of embodiments 1-4, wherein the plant material and solution of a) is at a ratio of about 3:1.
19. The process of any one of embodiments 1-4, wherein the plant material and solution of a) is at a ratio of about 2:1.
20. The process of any one of embodiments 1-4, wherein the plant material and solution of a) is at a ratio of about 1:1.
21. The process of any one of embodiments 1-4, wherein the lysing of the plant material comprises adding one or more divalent ion(s) to the lysate and/or filtrate and/or adding chitosan to the lysate and/or filtrate.
22. The process of any one of embodiments 1-4, wherein the lysing of the plant material comprises adding calcium ions to the lysate.
23. The process of any one of embodiments 1-4, wherein the lysing of the plant material comprises adding calcium chloride to the lysate.
24. The process of any one of embodiments 1-4, wherein the plant material is lysed chemically, mechanically, and/or enzymatically.
25. The process of any one of embodiments 1-4, wherein the plant material is lysed chemically.
26. The process of any one of embodiments 1-4, wherein the plant material is lysed chemically using one or more detergents.
27. The process of any one of embodiments 1-4, wherein the plant material is lysed chemically using CHAPS.
28. The process of any one of embodiments 1-4, wherein the plant material is lysed enzymatically using one or more enzymes.
29. The process of any one of embodiments 1-4, wherein the plant material is lysed using cellulase or pectinase.
30. The process of any one of embodiments 1-4, wherein the plant material is lysed mechanically.
31. The process of any one of embodiments 1-4, wherein the plant material is lysed mechanically using a blender.
32. The process of any one of embodiments 1-4, wherein the plant material is lysed mechanically using mills, homogenizers, microfluidizers, mechanical pressure, or a Stephan cutter.
33. The process of any one of embodiments 1-4, wherein the plant material is lysed mechanically using a press, a sonicator, a disintegrator, using a pulse electric field, using nitrogen burst, using ultrasonic energy, or by freezing.
34. The process of any one of embodiments 1-4, wherein the plant material is lysed mechanically using at least one mill.
35. The process of any one of embodiments 1-4, wherein the plant material is lysed mechanically using at least two different types of mills.
36. The process of any one of embodiments 1-4, wherein the separating of c) is performed with a screw press, a decanter or a centrifuge.
37. The process of any one of embodiments 1-4, wherein the separating of c) is performed using a disk stack centrifuge, a continuous centrifuge, or a basket centrifuge.
38. The process of any one of embodiments 1-4, wherein the separating of c) is performed using filtration.
39. The process of any one of embodiments 1-4, wherein the separating of c) is performed using a press.
40. The process of any one of embodiments 1-4, wherein the separating of c) is performed using filtration.
41. The process of any one of embodiments 1-4, wherein the separating of c) is performed using gravity settling.
42. The process of any one of embodiments 1-4, wherein the separating of c) is performed using sieving.
43. The process of embodiment 1, wherein the first set temperature of d) is no more than about 80° C.
44. The process of embodiment 1, wherein the first set temperature of d) is no more than about 65° C.
45. The process of embodiment 1, wherein the first set temperature of d) is no more than about 55° C.
46. The process of embodiment 1, wherein the first set temperature of d) is no more than about 50° C.
47. The process of embodiment 1, wherein the second set temperature of d) is no more than about 25° C.
48. The process of embodiment 1, wherein the second set temperature of d) is no more than about 15° C.
49. The process of embodiment 1, wherein the second set temperature of d) is no more than about 10° C.
50. The process of embodiment 1, wherein heating to a first set temperature of d) takes no more than about 15 min.
51. The process of embodiment 1, wherein heating to a first set temperature of d) takes no more than about 5 min.
52. The process of embodiment 1, wherein cooling to a second set temperature of d) takes no more than about 15 min.
53. The process of embodiment 1, wherein cooling to a second set temperature of d) takes no more than about 5 min.
54. The process of embodiment 2, wherein the one or more salts of d) comprise one or more calcium salts, one or more magnesium salts, one or more beryllium salts, one or more zinc salts, one or more cadmium salts, one or more copper salts, one or more iron salts, one or more cobalt salts, one or more tin salts, one or more strontium salts, one or more barium salts, and/or one or more radium salts.
55. The process of embodiment 2, wherein the one or more salts of d) comprise potassium phosphate and/or calcium chloride.
56. The process of embodiment 2, wherein the one or more salts of d) is added at a concentration of 5 mM to 2 M.
57. The process of any one of embodiments 1-4, wherein the flocculant is an alkylamine epichlorohydrin, polydimethyldiallylammonium chloride, a polysaccharide, a polyamine, starch, aluminum sulphate, alum, polyacrylamide, polyacromide, or polyethyleneimine.
58. The process of any one of embodiments 1-4, wherein the flocculant is chitosan.
59. The process of any one of embodiments 1-4, wherein the flocculant is activated chitosan.
60. The process of any one of embodiments 1-4, wherein the flocculant is 1-20% w/w activated chitosan in solution.
61. The process of any one of embodiments 1-4, wherein the adsorbent of e) is a resin.
62. The process of any one of embodiments 1-4, wherein the adsorbent of e) is activated carbon, activated charcoal, or activated coal.
63. The process of any one of embodiments 1-4, wherein the adsorbent of 3) is activated carbon that has a surface area in excess of 250 m/g, a weight average diameter of 1-1000 □m, an iodine number of 400-1,400 mg/g, a molasses number in the range of 100-550, and/or a Methylene Blue adsorption of at least 10 g/100 g.
64. The process of any one of embodiments 1-4, wherein the separation of f) is performed at no more than 25° C.
65. The process of any one of embodiments 1-4, wherein the separation of f) is performed at no more than 15° C.
66. The process of any one of embodiments 1-4, wherein the separation of f) is performed at no more than 10° C.
67. The process of any one of embodiments 1-4, wherein the separation of f) is performed using filtration.
68. The process of any one of embodiments 1-4, wherein the separation of f) is performed using a press, using gravity settling, or by sieving.
69. The process of any one of embodiments 1-4, wherein the separation of f) is performed using a centrifuge, or a decanter, or by microfiltration.
70. The process of any one of embodiments 1-4, wherein all steps of the process except for e) are performed at no more than 25° C.
71. The process of any one of embodiments 1-4, wherein all steps of the process except for e) are performed at no more than 15° C.
72. The process of any one of embodiments 1-4, wherein all steps of the process except for e) are performed at no more than 10° C.
73. The process of any one of embodiments 1-4, wherein the filtering of g) is performed with a membrane filter.
74. The process of any one of embodiments 1-4, wherein the filtering of g) is performed with a 0.7 □m membrane filter.
75. The process of any one of embodiments 1-4, wherein the filtering of g) is performed with a 0.2 □m membrane filter.
76. The process of any one of embodiments 1-4, wherein the filtering of g) is performed with diatomaceous earth and/or activated carbon.
77. The process of any one of embodiments 1-4, wherein the filtering of g) is performed with up to about 10% activated carbon.
78. The process of any one of embodiments 1-4, wherein the filtering of g) is performed with up to about 2% activated carbon.
79. The process of any one of embodiments 1-4, wherein the filtering of g) is performed with a 0.2 μm membrane filter and about 2% activated carbon.
80. The process of any one of embodiments 73-79, further comprising, after g), filtering the filtrate of g) through a 0.2 μm membrane filter.
81. The process of any one of embodiments 1-4, wherein one or more liquid phases and/or one or more filtrates comprise one or more anti-foaming agents and/or one or more defoaming agents.
82. The process of any one of embodiments 1-4, wherein one or more liquid phases and/or one or more filtrates is filtered to remove small solids and/or microorganisms.
83. The process of any one of embodiments 1-4, wherein one or more liquid phases and/or one or more filtrates is sterilized.
84. The process of any one of embodiments 1-4, further comprising concentrating the filtrate.
85. The process of embodiment 84, wherein concentrating the filtrate is performed by ultrafiltration.
86. The process of embodiment 85, wherein the ultrafiltration is through polyethersulfone, polypropylene, polyvinylidene fluoride, polyacrylonitrile, cellulose acetate, or polysulfone.
87. The process of embodiment 85, wherein the ultrafiltration performed using an ultrafiltration filter with a cut-off of no more than 100 kDa.
88. The process of embodiment 85, wherein the ultrafiltration performed using an ultrafiltration filter with a cut-off of no more than 50 kDa.
89. The process of embodiment 85, wherein the ultrafiltration performed using an ultrafiltration filter with a cut-off of no more than 10 kDa.
90. The process of any one of embodiments 1-4, wherein the yield of the purified protein is at least about 10% the soluble protein in the liquid phase of step c).
91. The process of any one of embodiments 1-4, wherein the yield of the purified protein is at least about 20% the soluble protein in the liquid phase of step c).
92. The process of any one of embodiments 1-4, wherein the yield of the purified protein is at least about 25% the soluble protein in the liquid phase of step c).
93. The process of any one of embodiments 1-4, wherein the purity of the purified protein is at least about 40%.
94. The process of any one of embodiments 1-4, wherein the purity of the purified protein is at least about 60%.
95. The process of any one of embodiments 1-4, wherein the purity of the purified protein is at least about 80%.
96. The process of any one of embodiments 1-95, wherein the weight ratio of chlorophyll to protein in the purified protein preparation is less than about 1:1000, about 1:1500, about 1:2000, or about 1:2500.
97. The process of any one of embodiments 1-96, wherein one or more agent(s) in the purified protein preparation that imparts or is associated with one or more organoleptic properties are reduced or removed relative to the source plant material.
98. The process of any one of embodiments 1-96, wherein one or more agent(s) in the purified protein preparation that imparts or is associated with one or more organoleptic properties are reduced by 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% relative to the source plant material.
99. The process of any one of embodiments 1-96, wherein the purified protein preparation is essentially odorless.
100. The process of any one of embodiments 1-96, wherein the purified protein preparation is odorless.
101. The process of any one of embodiments 1-100, wherein the purified protein preparation is essentially neutral-tasting.
102. The process of any one of embodiments 1-100, wherein the purified protein preparation is neutral-tasting.
103. The process of any one of embodiments 1-102, wherein the protein is RuBisCo.
104. The process of any one of embodiments 1-103, wherein the plant material is from Lemna.
105. The process of any one of embodiments 1-103, wherein the plant material is from Lemnoideae.
106. A product made by the process of any one of embodiments 1-105.
107. A food comprising a purified protein preparation from a plant material, wherein the protein preparation contains no more than 80% impurities.
108. The food of embodiment 107, wherein the protein preparation comprises RuBisCo.
109. The food of embodiments 107 or 108, wherein the plant material is from Lemna.
110. The food of embodiments 107 or 108, wherein the plant material is from Lemnoideae.

In some aspects of the disclosure, the plant material is washed before the process is started. In some embodiments, the reducing agent in a) is 2-mercaptoethanol (BME), 2-mercaptoethylamine-HCL, sodium metabisulfite, cysteine hydrochloride, dithiothreitol (DTT), glutathione, cysteine, tris(2-carboxyethyl)phosphine (TCEP), ferrous ion, nascent hydrogen, sodium amalgam, oxalic acid, formic acid, magnesium, manganese, phosphorous acid, potassium, and sodium. In some embodiments, the reducing agent is a sulfite. In some embodiments, the sulfite is sodium sulfite, magnesium sulfite, or sodium metabisulfite. In some embodiments, the sulfite is sodium bisulfate.

In some embodiments, the pH of the solution in a) is about pH 5.0 to about pH 9.0. In some embodiments, the pH of the solution is about pH 6.0 to about pH 7.6. In some embodiments, the pH of the solution is about 6.8.

In some embodiments, the plant material and solution of a) is at a ratio of about 6:1. In some embodiments, the plant material and solution of a) is at a ratio of about 3:1. In some embodiments, the plant material and solution of a) is at a ratio of about 2:1. In some embodiments, the plant material and solution of a) is at a ratio of about 1:1.

In some aspects of the disclosure, the plant material is lysed mechanically. In some embodiments the plant material is lysed mechanically using a blender. In some embodiments, the plant material is lysed mechanically using mills, homogenizers, microfluidizers, mechanical pressure or Stephan cutter.

In some aspects of the disclosure, the separating of c) is performed with a screw press, a decanter or a centrifuge.

In some aspects of the disclosure, the first set temperature of d) is no more than about 80° C. In some aspects of the disclosure, the first set temperature of d) is no more than about 65° C. In some embodiments, the first set temperature of d) is no more than about 55° C. In some embodiments, the first set temperature of d) is no more than about 50° C. In some embodiments, the second set temperature of d) is no more than about 25° C. In some embodiments, the second set temperature of d) is no more than about 15° C. In some embodiments, the second set temperature of d) is no more than about 10° C. In some embodiments, heating to a first set temperature of d) takes no more than about 15 min. In some embodiments, heating to a first set temperature of d) takes no more than about 5 min. In some embodiments, cooling to a second set temperature of d) takes no more than about 15 min. In some embodiments, cooling to a second set temperature of d) takes no more than about 5 min.

In some aspects of the disclosure, the one or more salts of d) comprise potassium phosphate and/or calcium chloride. In some aspects of the disclosure, the one or more salts of d) is added at a concentration of 5 mM to 2 M.

In some aspects of the disclosure, the flocculant is chitosan. In some aspects of the disclosure, the flocculant is activated chitosan. In some embodiments, the flocculant is 1-20% w/v activated chitosan in solution. In some embodiments, the adsorbent of e) is activated carbon, activated charcoal, or activated coal. In some embodiments, the adsorbent is a hydrophobic adsorbent.

In some aspects of the disclosure, separation of f) is performed at no more than 25° C. In some embodiments, the separation of f) is performed at no more than 15° C. In some embodiments, the separation of f) is performed at no more than 10° C. In some embodiments, the separation of f) is performed using a centrifuge, or a decanter, or by microfiltration.

In some aspects of the disclosure, all steps of the process except for e) are performed at no more than 25° C. In some embodiments, all steps of the process except for e) are performed at no more than 15° C. In some embodiments, all steps of the process except for e) are performed at no more than 10° C.

In some aspects of the disclosure, the filtering of g) is performed with a membrane filter. In some embodiments, the filtering of g) is performed with a 0.7 μm membrane filter. In some embodiments, the filtering of g) is performed with a 0.2 μm membrane filter. In some embodiments, wherein the filtering of g) is performed with diatomaceous earth and/or an activated carbon. In some embodiments, the filtering of g) is performed with up to about 10% activated carbon. In some embodiments, the filtering of g) is performed with up to about 2% activated carbon. In some embodiments, the filtering of g) is performed with a 0.2 μm membrane filter and a 2% activated carbon. In some embodiments, the process further comprises, after g), filtering the filtrate of g) through a 0.2 μm membrane filter. In some embodiments, the process further comprises concentrating the filtrate. In some embodiments, concentrating the filtrate is performed by diafiltration. In some embodiments, concentrating the filtrate is performed by ultrafiltration. In some embodiments, the ultrafiltration performed using an ultrafiltration filter with a cut-off of no more than 100 kDa. In some embodiments, the ultrafiltration performed using an ultrafiltration filter with a cut-off of no more than 50 kDa. In some embodiments, the ultrafiltration performed using an ultrafiltration filter with a cut-off of no more than 10 kDa.

In some aspects of the disclosure, the yield of the purified protein is at least about 10% of the soluble protein in the liquid phase of step c). In some embodiments, the yield of the purified protein is at least about 20% the soluble protein in the liquid phase of step c). In some embodiments, the yield of the purified protein is at least about 25% the soluble protein in the liquid phase of step c). In some embodiments, the purity of the purified protein is at least about 40%. In some embodiments, the purity of the purified protein is at least about 60%. In some embodiments, the purity of the purified protein is at least about 80%.

In some aspects of the disclosure, the protein is RuBisCo. In some embodiments, the plant material is from Lemna. In some embodiments, the plant material is from Lemnoideae.

Also disclosed herein are products made by the processes disclosed.

Also disclosed herein are foods comprising a purified protein preparation from a plant material, wherein the protein preparation contains no more than 80% impurities.

### BRIEF DESCRIPTION OF FIGURES

FIG. 1: Flow chart of one embodiment of the process.

FIG. 2: Flow chart of a second embodiment of the process.

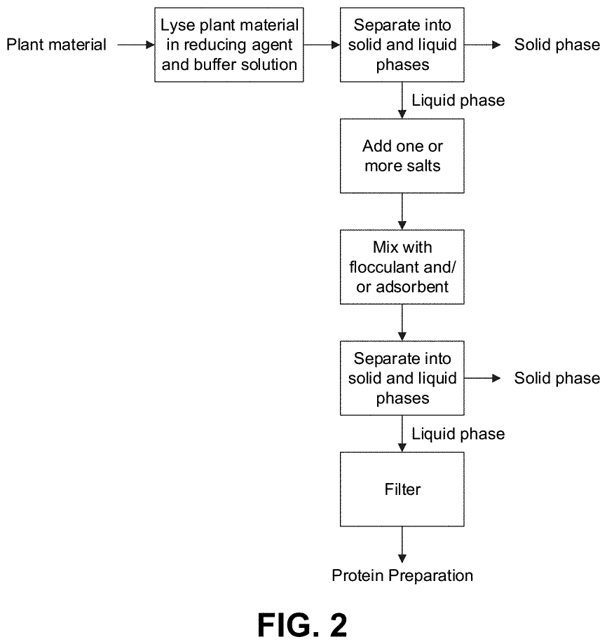

FIG. 3: Flow chart of a third embodiment of the process.

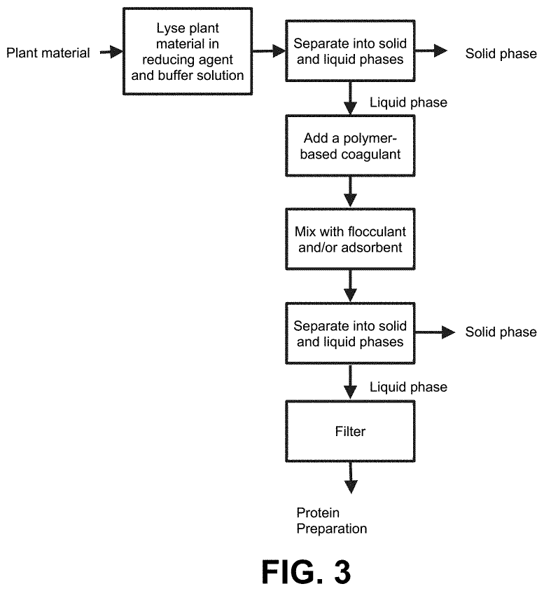

FIG. 4: Flow chart of a fourth embodiment of the process.

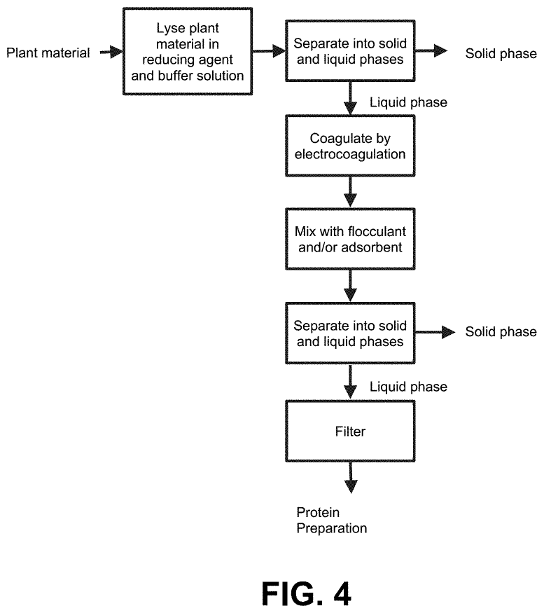

FIG. 5: Depiction of Fractions 1, 2, 3, and 4 of Example 5 after microfiltration.

FIG. 6: Depiction of Fractions 4, 3, 2, and 1 of Example 5 after microfiltration.

FIG. 7A: Depiction of Fractions of Example 5.

FIG. 7B: Depiction of Fractions of Example 5.

FIG. 8: Depiction of samples of Fractions of Example 6 after calcium chloride and phosphate addition and benchtop centrifugation.

FIG. 9: Depiction of samples of Fractions 1-6 of Example 6 after removal of activated carbon and chitosan.

FIG. 10: Depiction of results of SDS-PAGE Coomassie staining analysis for samples of Fractions of Example 6.

FIG. 11: Depiction of SDS-PAGE gel for various samples of Example 7.

FIG. 12: Depiction of Fractions of Example 7 after removal of activated carbon and chitosan.

FIG. 13: Depiction of SDS-PAGE gel for various samples of Example 8.

FIG. 14: Depiction of SDS-PAGE gel for various samples of Example 9.

FIG. 15: Depiction of Fractions 1-5 of Example 10 after microfiltration.

FIG. 16: Depiction of a chromatogram of final protein product and protein standard.

FIG. 17: Depiction of an SDS-PAGE gel of final plant protein product.

FIG. 18: Depiction of an absorbance spectrum of materials of Example 16

### DETAILED DESCRIPTION

The disclosed processes and compositions may be understood more readily by reference to the following detailed description taken in connection with the accompanying figures, which form a part of this disclosure.

Throughout this text, the descriptions refer to processes and compositions made by the processes. Where the disclosure discloses or claims a feature or embodiment associated with a composition, such a feature or embodiment is equally applicable to the process of making the composition. Likewise, where the disclosure discloses or claims a feature or embodiment associated with a process of making a composition, such a feature or embodiment is equally applicable to the composition. When a range of values is expressed, it includes embodiments using any particular value within the range. Further, reference to values stated in ranges includes each and every value within that range. When values are expressed as approximations by use of the antecedent “about” it will be understood that the particular value forms another embodiment. The use of “or” will mean “and/or” unless the specific context of its use dictates otherwise. All references cited herein are incorporated by reference in their entirety for any purpose. Where a reference and the specification conflict, the specification will control. It is to be appreciated that certain features of the disclosed processes and compositions, which are, for clarity, disclosed herein in the context of separate embodiments, may also be provided in combination in a single embodiment. Conversely, various features of the disclosed processes and compositions that are, for brevity, disclosed in the context of a single embodiment, may also be provided separately or in any sub-combination.

As used herein, the singular forms “a,” “an,” and “the” include plural forms unless the context clearly dictates otherwise. The term “about” or “approximately,” when used in the context of numerical values and ranges, refers to values or ranges that approximate or are close to the recited values or ranges such that the embodiment may perform as intended, up to about plus or minus 10%, as is apparent to the skilled person from the teachings contained herein. In some embodiments, about means plus or minus 10% of a numerical amount.

Disclosed herein are processes for making a protein preparation from a plant material. As used herein, the term “plant” refers to an organism belonging to the kingdom Plantae. Examples of plants suitable for use in the disclosed processes include trees, herbs, bushes, grasses, vines, ferns, mosses, and green algae. The term “plant material” refers to any biomass derived from a plant. Plant material may be derived from any part of a plant, e.g., stem, root, fruit, leaves, or seeds. In some embodiments, plant material is derived from leaves. In some embodiments, plant material is derived from stems. The plant material may also be obtained from one or more species of plants. For instance, in some embodiments, plant material may be derived from duckweed, algae, sugar beet leaves, spinach, kale, beet, chard, sugar beet, sea beet, Mangel beet, soy, or tobacco. In some embodiments, plant material is derived from duckweed. In some embodiments, plant material is derived from Lemna. In some embodiments, plant material is derived from Lemnoideae.

As used herein, the term “protein” refers to a compound comprised of amino acid residues covalently linked by peptide bonds. A protein typically contains at least two amino acids or amino acid variants, and no limitation is placed on the maximum number of amino acids that can comprise a protein sequence. The term “protein preparation” refers to an isolate of proteins, wherein the proteins has been substantially separated from non-protein components of a mixture. The “purity” of a protein preparation refers to the amount of protein relative to the total amount of preparation. In some embodiments, the purity of the protein preparation is expressed as a percentage of the total dry mass. In some embodiments, a protein preparation comprises at least about 40%, 45%, 50%, 55%, 60%, 65%, 70%, 75%, 80%, 85%, 90%, 95%, 97%, or 99% protein. In some embodiments, the purity of the protein preparation is at least about 40%, 45%, 50%, 55%, 60%, 65%, 70%, 75%, 80%, 85%, 90%, 95%, 97%, or 99% protein. A protein preparation may comprise one or more types of protein and may comprise different sizes of the same protein. For instance, in some embodiments, the protein preparation may comprise edestin, gluten, legumin or vicilin. In some embodiments, the protein preparation comprises RuBisCo. The processes disclosed herein separates proteins from other compounds found in plant material. For example, the process may remove chlorophyll, volatilized chemical compounds, acids, bases, sugars, salts, and/or lipids.

In some embodiments, the processes disclosed herein remove chlorophyll from plant material, producing protein preparations that are dechlorophyllized. For instance, in some embodiments, the weight ratio of chlorophyll to protein in the protein preparation is less than about 1:1000, 1:1500, 1:2000, or 1:2500.

In some embodiments, the processes disclosed herein reduce or remove one or more agent(s) that imparts or is associated with one or more organoleptic properties in the purified protein preparation. Non-limiting examples of such organoleptic properties include odor (e.g., off-odor or undesirable odor) and taste (e.g., off-taste or undesirable taste). In some embodiments, the processes disclosed herein reduce the one or more agent(s) by 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% relative to the source plant material. In some embodiments, the processes disclosed herein completely reduce the one or more agent(s). In some embodiments, the processes disclosed herein reduce or remove one or more agent(s) that imparts or is associated with odor and produce protein preparations that are odorless or essentially odorless. In some embodiments, the processes disclosed herein reduce or remove one or more agent(s) that imparts or is associated with taste and produce neutral-tasting protein preparations or essentially neutral-tasting protein preparations. In some embodiments, the processes disclosed herein reduce or remove one or more agent(s) that imparts or is associated with odor and/or taste and produce protein preparations that are odorless and neutral-tasting, essentially odorless and neutral-tasting, odorless and essentially neutral-tasting, or essentially odorless and essentially neutral-tasting. In some embodiments, the agent is a volatile compound. In some embodiments, the agent is a non-volatile compound. In some embodiments, the agent is a polyphenol, polyphenol oxidase, lipoxygenase, a phenol, a lipid, an alcohol, an aldehyde, a sulfide, a peroxide, a terpene, an albumin (e.g., a lectin or a protease inhibitor), a substrate for an oxidative enzymatic activity (e.g., a fatty acid, such as (C14:0 (methyl myristate), C15:0 (methyl pentadecanoate), C16:0 methyl palmitate, C16:1 methyl palmitoleate, C17:0 methyl heptadecanoate, C18:0 methyl stearate, C18:1 methyl oleate, C18:2 methyl linoleate, C18:3 methyl alpha linoleate, C20:0 methyl eicosanoate, and C22:0 methyl behenate, and/or an enzyme that reacts with a lipid substrate. In some embodiments, the purified protein preparation has a reduced oxidative enzymatic activity relative to the source of the protein. In some embodiments, the purified protein preparation has a 5%, 10%, 15%, 20%, or 25% reduction in oxidative enzymatic activity relative to the source of the protein. In some embodiments, the source of the protein is RuBisCo, and the purified protein preparation has a 5%, 10%, 15%, 20%, or 25% reduction in oxidative enzymatic activity relative to RuBisCo. In some embodiments, the oxidative enzymatic activity is lipoxygenase activity. In some embodiments, the purified protein preparation has lower oxidation of lipids or residual lipids relative to the source of the protein due to reduced lipoxygenase activity.

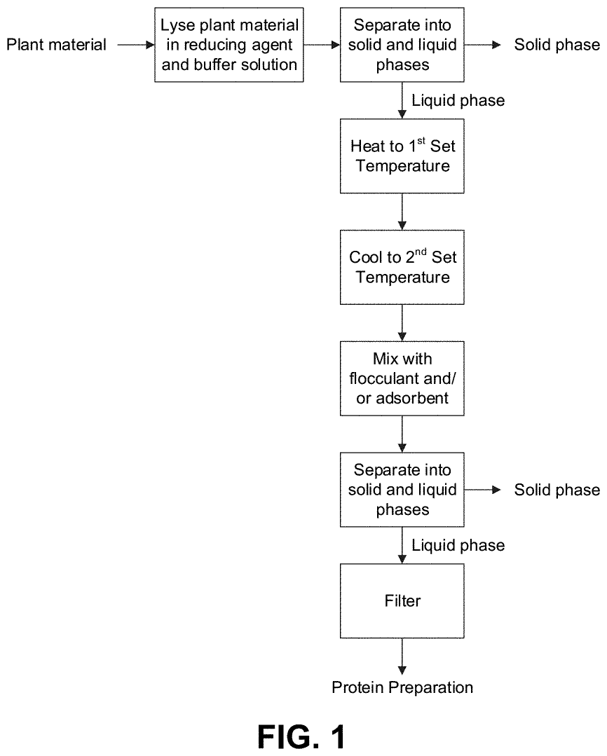

In one aspect of the disclosure, a process (FIG. 1) for making a purified protein preparation from a plant material, comprises the steps of:
a) providing the plant material in a solution comprising a reducing agent;
b) lysing the plant material;
c) separating the lysed plant material into a solid phase and a liquid phase, wherein the liquid phase contains soluble protein and chlorophyll;
d) coagulating the chlorophyll in the liquid phase by heating it to a first set temperature in no more than about 30 min, then cooling it to a second set temperature in no more than about 30 min, wherein the cooling is initiated when the liquid phase reaches the first set temperature.
e) contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) separating the flocculated mixture of e) into a solid phase and a liquid phase; and
g) filtering the liquid phase of f) to yield a filtrate containing a purified protein.

In some embodiments, the plant material is harvested and cleaned before the process is started. For instance, in some embodiments, the plant material is chemically washed before the process is started. In some embodiments, the plant material is washed with water before the process is started. The plant material may also undergo multiple rounds of washes before the process is started.

In some embodiments, the plant material is mixed in a solution comprising a reducing agent. Examples of reducing agents suitable for use in the disclosed processes include, but are not limited to, 2-mercaptoethanol (BME), 2-mercaptoethylamine-HCL, sodium metabisulfite, cysteine hydrochloride, dithiothreitol (DTT), glutathione, cysteine, tris(2-carboxyethyl)phosphine (TCEP), ferrous ion, nascent hydrogen, sodium amalgam, oxalic acid, formic acid, magnesium, manganese, phosphorous acid, potassium, and sodium. In some embodiments, the plant material is mixed in a solution comprising more than one reducing agent. In some embodiments, the reducing agent is a sulfite. In some embodiments, the reducing agent is at least one of sodium sulfite, magnesium sulfite, or sodium metabisulfite. In some embodiments, the reducing agent is sodium bisulfate. Without wishing to be bound by theory, it is believed that reducing agents act to regulate and/or inhibit the activity of polyphenol oxidase.

The solution comprising the reducing agent may be formulated to improve the stability of its components. For example, the pH, ionic strength or temperature of the solution may be adjusted. In some embodiments, the solution may comprise buffering agents. Examples of buffering agents for use in the disclosed processes include, but are not limited to, alkali metals (e.g., Na⁺ or K⁺), NaCl, ammonium ions (NH₄), nitrates, acetates (e.g., sodium acetate), chlorates, perchlorates (NO³⁻, C₂H₃O²⁻, ClO³⁻, ClO⁴⁻), binary compounds of halogens with metals (e.g., Cl⁻, Br⁻, or I⁻), sulfates (SO₄²⁻), ammonium sulfate, hydroxides of alkali earth metals (e.g., OH⁻, Ca²⁺, or Sr²⁺), sulfides (S²⁻), hydroxides (OH−), carbonates (e.g., sodium carbonate), oxalates, chromates (CO₃²⁻, C₂O₄²⁻, CrO₄²⁻,) phosphates (PO₄³⁻) (e.g., sodium phosphate, monopotassium phosphate (KH₂PO₄), dipotassium phosphate (K₂HPO₄), monosodium phosphate (NaH₂PO₄), disodium phosphate (Na₂HPO₄), ammonium phosphate (NH₄)₃PO₄, calcium phosphate (Ca₃(PO₄)₂), magnesium phosphate, monomagnesium phosphate, dimagnesium phosphate, and trimagnesium phosphate), Tris-HCl, HEPES, ACES, ADA, BES, Imidazole-HCl, MES, MOPS, MOPSO, PIPES, TES, Bis-Tris, Tricine, Bicine, 3-{[tris(hydroxymethyl)methyl]amino}propanesulfonic acid, N,N-bis(2-hydroxyethyl)glycine, tris(hydroxymethyl)methylamine, N-tris(hydroxymethyl)methylglycine, 3-[N-Tris(hydroxymethyl)methylamino]-2-hydroxypropanesulfonic Acid, 4-2-hydroxyethyl-1-piperazineethanesulfonic acid, 2-{[tris(hydroxymethyl)methyl]amino}ethanesulfonic acid, 3-(N-morpholino)propanesulfonic acid, piperazine-N,N′-bis(2-ethanesulfonic acid), dimethylarsinic acid, sodium citrate, saline sodium citrate, 2-(N-morpholino)ethanesulfonic acid, cholamine chloride, acetaminoglycine, tricine, glycinamide and ammonium carbonate

In some embodiments, solutions for use in the disclosed processes comprise chelating agents. Examples of chelating agents for use in the disclosed processes include, but are not limited to, chloride, cyanide, organic acids (including but not limited to citric acid, glycolic acid, lactic acid, malic acid, malonic acid, oxalic acid, and succinic acid), deferoxamine, deferiprone, deferasirox, penicillamine, honey, sodium pyrophosphate, sodium hexametaphosphate, sporix, BAL, EDTA, dexrazoxane, Prussian Blue, ALA, BAPTA, DTP A, DMPS, DMSA, EGTA, ribose, deoxyribose, glucose, fructose, glucosamine, sucrose, lactose, maltose, cellulose, starch, pectins, gums, alginic acid, chitin, chitosans, lactic acid, pyruvic acid, citric acid, acetic acid, lipids, monoglyceride, diglyceride, triglyceride, phosphyatidylcholine, phosphatidylethanolamine, ceramide, sphingomyelin, xanthophylls, vitamin A, cortisone, cortisole, cholic acid, deoxycholic acid, taurocholic acid, glycine, alanine, valine, leucine, isoleucine, phenylalanine, tryptophan, serine, threonine, tyrosine, aspartic acid, glutamic acid, lysine, arginine, asparagine, glutamine, histidine, cysteine, methionine, proline, histamine, adrenaline, insuline, ATP, NAD, FMN, FAD, Coenzyme A, DNA, RNA, carbonate, bicarbonate, cyanides, glycolic acid, oxalic acid, lactic acid, citric acid, orthophosphates, pyrophosphates, metaphosphates, polyphosphates, phytic acid, MDP, HMDP, HEDP, hemoglobin, chlorophyll, plant alkaloids, anthocyanins, tannins, sulfates, sulfonic acids, chondroitin sulfates, vitamin B12, ascorbic acid, and water.

In some embodiments, solutions for use in the disclosed processes comprise protease inhibitors. Examples of protease inhibitors for use in the disclosed processes include, but are not limited to, PMSF, sodium fluoride, beta-glycerophosphate, sodium pyrophosphate, leupeptin, and E-64.

In some embodiments, the pH of the solution is about 5.0 to about 9.0. In some embodiments, the pH of the solution is about 6.5 to about 7.5. In some embodiments, the pH of the solution is about 7.5. In some embodiments, the pH of the solution is about 6.5 to about 7.0.

The plant material and the solution comprising the reducing agent may be mixed in proportions that increase the accessibility of the plant material to the reducing agent. For example, the plant material and the solution comprising the reducing agent may be fixed at a ratio of about 6:1, 3:1, 2:1, or 1:1.

As used herein, the term “lysing” refers to breaking up of the cells from the plant material and exposing the contents of the cell. For instance, lysing may comprise breaking the cell wall, disrupting the plasma membrane, and/or exposing the cytoplasm. Methods for lysing plant material are known in the art, and may comprise mechanical, chemical, and/or enzymatic lysis. In some embodiments, the plant material is lysed mechanically. Examples of mechanical lysis suitable for use in the disclosed processes include, but are not limited to, mechanical agitation, pressure, grinding, squeezing, and shearing. In some embodiments, the plant material is lysed mechanically using a blender. In some embodiments, the plant material is lysed mechanically using a mill, e.g., by knife mill, high shear mill, colloid mill, ball mill, Boston shear mill, hammer mill, grinding mill, Rietz mill, wet mill or high shear mill. In some embodiments, the plant material is lysed mechanically using at least two different types of mills (e.g., via tandem milling). Without wishing to be bound by theory, it is believed that, in some embodiments, mechanically lysing plant material using at least two different types of mills results in more effective lysing of the plant material. In some embodiments, the plant material is lysed mechanically using a sonicator, using nitrogen burst, using ultrasonic energy, or by freezing. In some embodiments, the plant material is lysed mechanically using a press (e.g., a screw press or a French press). In some embodiments, the plant material is lysed mechanically using a homogenizer (e.g., a high-pressure homogenizer or a microfluidizer). In some embodiments, the plant material is lysed mechanically using a disintegrator. In some embodiments, the plant material is lysed mechanically using a pulse electric field (PEF). In some embodiments, the plant material is lysed mechanically using mechanical pressure. In some embodiments, the plant material is lysed mechanically using one or more of the techniques for mechanical lysis disclosed herein.

In some embodiments, the plant material is lysed chemically. In some embodiments, the plant material is lysed chemically using one or more detergents. In some embodiments, the one or more detergents are ionic detergents. In some embodiments, the one or more detergents are cationic detergents. In some embodiments, the one or more detergents are anionic detergents. In some embodiments, the one or more detergents include sodium dodecyl sulfate (SDS). In some embodiments, the one or more detergents are non-ionic detergents, such as Triton X-100, NP-40, digitonin, and/or saponin. In some embodiments, the one or more detergents are zwitterionic detergents, such as Triton, NP, Brij, Tween, octyl-beta-glucoside, octylthioglucoside, SDS, CHAPS, and/or CHAPSO. In some embodiments, the one or more detergents are hypotonic detergents. In some embodiments, the one or more detergents are hypertonic detergents. In some embodiments, the one or more detergents are isotonic detergents. In some embodiments, the plant material is lysed chemically using one or more of the techniques for chemical lysis disclosed herein.

In some embodiments, the plant material is lysed enzymatically using one or more enzymes. In some embodiments, the one or more enzymes include cellulase. In some embodiments, the one or more enzymes include pectinase.

In some embodiments, the plant material is lysed chemically and mechanically. In some embodiments, the plant material is lysed chemically and enzymatically. In some embodiments, the plant material is lysed mechanically and enzymatically. In some embodiments, the plant material is lysed chemically, mechanically, and enzymatically.

In some embodiments, the lysing of the plant material includes adding one or more divalent ion(s) to the lysate. In some embodiments, the lysing of the plant material comprises adding chitosan to the lysate. In some embodiments, the lysing of the plant material comprises adding one or more divalent ion(s) to the lysate and adding chitosan to the lysate. In some embodiments, the lysing of the plant material comprises adding calcium ions to the lysate. In some embodiments, the lysing of the plant material comprises adding calcium ions to the lysate and adding chitosan to the lysate. In some embodiments, the lysing of the plant material comprises adding calcium chloride to the lysate. In some embodiments, the lysing of the plant material comprises adding calcium chloride to the lysate and adding chitosan to the lysate.

Separation of the lysed plant material into a solid phase and a liquid phase may be performed by any solid-liquid separation techniques known in the art. Examples of such separation techniques suitable for use in the disclosed processes include sieving, filtration, centrifugation and decanting. In some embodiments, separating the lysed plant material into a solid phase and a liquid phase is performed with a screw press, a decanter or a centrifuge. In some embodiments, separating the lysed plant material into a solid phase and a liquid phase is performed using a disk stack centrifuge, a decanter centrifuge, a continuous centrifuge, or a basket centrifuge. In some embodiments, separating the lysed plant material into a solid phase and a liquid phase comprises filtration, including but not limited to using a dead-end filtration system, using ultrafiltration, using a tangential flow filtration system, or using a plate filter. In some embodiments, separating the lysed plant material into a solid phase and a liquid phase comprises use of a press, including but not limited to a screw press, a French press, a belt press, a filter press, a fan press, a finisher press, or a rotary press. In some embodiments, separating the lysed plant material into a solid phase and a liquid phase comprises using gravity settling. In some embodiments, separating the lysed plant material into a solid phase and a liquid phase comprises sieving, including but not limited to using a circular vibratory separator or a linear/inclined motion shaker. In some embodiments, the liquid phase comprises soluble proteins and chlorophyll. In some embodiments, the solid phase comprises insoluble proteins, lignin, fibers, etc.

Separation of the lysed plant material into a solid phase and a liquid phase may yield materials that are useful in various applications, including but not limited to agricultural applications. For example, the liquid phase obtained from the separation of the lysed plant material may comprise soluble proteins, chlorophyll, phenolic compounds, cellular membranes (e.g., lipids), carbohydrates (including but not limited to pectin), nucleic acids, and/or light harvesting complexes/photosystems. For example, the solid phase obtained from the separation of the lysed plant material may comprise plant fiber(s), cellulose, hemicellulose, pectin, intact plant cells, cellular organelles, insoluble proteins, chlorophyll, and/or fats. In some embodiments, this solid phase may be used as, for example, an animal feed or as a biofuel. In some embodiments, this solid phase may contain levulinic acid, which is a precursor in the manufacture of biofuels. In some embodiments, chlorophyll obtained from the solid phase obtained from the separation of the lysed plant material may be used in, for example, cosmetic applications, as a dye, and/or in human and/or animal nutrition.

The process for making the purified protein preparation may also comprise a step of coagulating components that are undesired (e.g., components that are not RuBisCO) using heat treatment, leaving the desired protein component (e.g., RuBisCO) in the liquid phase. Without being bound by theory, heating causes conformational unfolding of amino acid chains, resulting in aggregation of some proteins. Depending on their amino acid sequence and conformational states, proteins have different unfolding temperatures, above which they will begin to unfold and aggregate. With carefully controlled heating and cooling conditions, proteins with unfolding temperatures lower than that of the desired protein product may be coagulated. In some embodiments, the heating is conducted under mild conditions to prevent the protein of interest from also aggregating. In some embodiments, the liquid phase is heated to a first set temperature. In some embodiments, the first set temperature is no more than about 40° C., 45° C., 50° C., 55° C., 60° C., 65° C., 70° C., 75° C. or 80° C. In some embodiments, the heating is performed rapidly. In some embodiments, the heating to the first set temperature takes no more than about 30 min. In some embodiments, heating to the first set temperature takes no more than 15 min. In some embodiments, heating to the first set temperature takes no more than 5 min. In some embodiments, the liquid phase is cooled to a second set temperature after heating to the first set temperature. In some embodiments, the second set temperature is no less than about 30° C., 25° C., 20° C., 15° C., 10° C., or 5° C. In some embodiments, the cooling is initiated immediately upon reaching the first set temperature. In some embodiments, the cooling is performed rapidly. In some embodiments, cooling to a second set temperature takes no more than about 30 min. In some embodiments, cooling to a second set temperature takes no more than about 15 min. In some embodiments, cooling to a second set temperature takes no more than about 5 min.

The process for making the purified protein preparation may also comprise a step of coagulating components that are undesired (e.g., components that are not RuBisCO) by addition of one or more salts, leaving the desired protein component (e.g., RuBisCO) in the liquid phase. In some embodiments, the salt is a calcium salt, a magnesium salt, a beryllium salt, a zinc salt, a cadmium salt, a copper salt, an iron salt, a cobalt salt, a tin salt, a strontium salt, a barium salt, a radium salt, or combinations thereof. In some embodiments, the salt is calcium chloride, calcium nitrate, or iron carbonate. In some embodiments, the salt added is potassium phosphate. In some embodiments, the salt added is calcium chloride. In some embodiments, the salts added are potassium phosphate and calcium chloride. In some embodiments, the one or more salt is added at a concentration of 5 mM to 2 M.

In some embodiments, the process for making the purified protein preparation may also comprise a step of coagulating components that are undesired (e.g., components that are not RuBisCO) by addition of one or more coagulants, leaving the desired protein component (e.g., RuBisCO) in the liquid phase, wherein the coagulant is a quaternary ammonia species, including but not limited to a protonated tertiary, secondary, or primary ammonium species. In some embodiments, the coagulant is selected from epiamines, polytannines, polyethylene imines, polylysines, and cationic polyacrylamides. In some embodiments, the coagulant is a polymer-based coagulant. In some embodiments, the polymer is zwitterionic. In some embodiments, the polymer is in the form of a solution or an emulsion. In some embodiments, the polymer is granular. In some embodiments, the polymer is a bead. In some embodiments, the polymer is uncharged. In some embodiments, the polymer has a charge density from less than 1 and up to 100% theoretical mole. In some embodiments, the polymer has a molecular weight is from 500 Daltons to 20 million Daltons. In some embodiments, the polymer has a molecular weight that is greater than 20 million Daltons.

In some embodiments, the process for making the purified protein preparation may also comprise a step of coagulating components that are undesired (e.g., components that are not RuBisCO) by electrocoagulation. In some embodiments of electrocoagulation, water passes through an electrocoagulation cell, wherein metal ion(s) are driven into the water, wherein, on a surface of a cathode, water is hydrolyzed into hydrogen gas and hydroxyl groups, wherein electrons flow freely to destabilize surface charges on suspended solids and emulsified oils, wherein large flocs form that entrain suspended solids, heavy metals, emulsified oils, and other contaminants, and wherein the flocs from removed from the water in downstream solids separation and/or filtration operation(s).

The process for making the protein preparation may also comprise a step of contacting the liquid phase with a flocculant and/or an adsorbent and mixing for a period of time sufficient to flocculate and/or adsorb chlorophyll in the liquid phase to the adsorbent, thereby forming a flocculated mixture. As used herein, the term “flocculant” refers to substance that is added to destabilize colloids and cause them to come out of a suspension. The term “adsorption” refers to adhesion of molecules to a solid surface or an “adsorbent.” The process of flocculation is well known in the art, and exemplary flocculants may include, but are not limited to, an alkylamine epichlorohydrin, polydimethyldiallylammonium chloride, a polysaccharide (e.g., chitosan), a polyamine, starch, aluminum sulphate, alum, polyacrylamide, polyacromide, or polyethyleneimine. In some embodiments, the flocculant is activated chitosan. In some embodiments, the flocculant is 1-20% w/v activated chitosan in solution. Methods to activate chitosan and dissolve chitosan in solution are well known in the art, and any method to prepare the activated chitosan in solution may be used. Exemplary methods may involve dissolving 1% chitosan in 20% acetic acid and 79% water. Adsorbents are known in the art, and exemplary adsorbents may include activated carbon, graphite, silica gel, zeolites, clay, polyethylene etc.

Exemplary adsorbents may also include resins. In some embodiments, resins for use in the disclosed processes are ion-exchange resins, including but not limited to strong cation exchangers, weak cation exchangers, strong anion exchangers, weak anion exchangers, mixed bed resins, chelating resins, and polymeric catalysts. In some embodiments, resins for use in the disclosed processes are size exclusion chromatography (SEC) resins, including but not limited to Sephacryl, Sephadex, Sepharose, and Superdex (GE Healthcare Bio-Sciences Corp, Westborough, Mass.). In some embodiments, resins for use in the disclosed processes have an affinity for a substrate analogue, an antibody-antigen, a polysaccharide (e.g., lectin), a complementary base sequence (e.g., a nucleic acid), a receptor (e.g., a hormone), avidin-biotin, calmodulin, poly-A, glutathione, proteins A and G, and/or metal ions. In some embodiments, resins for use in the disclosed processes have a hydrophobic interaction, such as resins comprising phenyl, butyl, octyl, hexyl, ether, and/or PPG.

In some embodiments, the adsorbent is activated carbon, activated charcoal, or activated coal. In some embodiments, the activated carbon has a surface area in excess of 250 m/g, a weight average diameter of 1-1000 □m, an iodine number of 400-1,400 mg/g, a molasses number in the range of 100-550, and/or a Methylene Blue adsorption of at least 10 g/100 g. In some embodiments, the liquid phase is contacted with a polymer. In some embodiments, the polymer is non-ionic. In some embodiments, the polymer is anionic. In some embodiments, the polymer is cationic. In some embodiments, the polymer is zwitterionic. In some embodiments, the polymer is in the form of a solution or an emulsion. In some embodiments, the polymer is granular. In some embodiments, the polymer is a bead. In some embodiments, the polymer is uncharged. In some embodiments, the polymer has a charge density from less than 1 and up to 100% theoretical mole. In some embodiments, the polymer has a molecular weight is from 500 Daltons to 20 million Daltons. In some embodiments, the polymer has a molecular weight that is greater than 20 million Daltons. Without being bound by theory, chlorophyll in the liquid phase may flocculate to form larger sized particles, which then adsorb to the surface of the activated carbon, charcoal or coal. The flocculated mixture comprises a solid phase comprising the flocculant, adsorbent, insoluble proteins and chlorophyll, and a liquid phase that comprises soluble proteins in solution.

The flocculated mixture may then be separated into a solid phase and a liquid phase. Separation of the flocculated mixture may be performed by any solid-liquid separation techniques known in the art. Examples of such separation techniques suitable for use in the disclosed processes include sieving, filtration, centrifugation and decanting. In some embodiments, separation of the flocculated mixture into a solid phase and a liquid phase is performed with a screw press, a decanter or microfiltration. In some embodiments, separation of the flocculated mixture into a solid phase and a liquid phase comprises centrifugation, such as the use of a decanter centrifuge, a disk stack centrifuge, or a continuous centrifuge. In some embodiments, separation of the flocculated mixture into a solid phase and a liquid phase comprises filtration, such as the use of a dead-end filtration system, ultrafiltration, the use of a tangential flow filtration system, or a plate filter. In some embodiments, separation of the flocculated mixture into a solid phase and a liquid phase comprises use of a press, such as a screw press, a French press, a belt press, a filter press, a fan press, a finisher press, or a rotary press. In some embodiments, separation of the flocculated mixture into a solid phase and a liquid phase comprises gravity settling. In some embodiments, separation of the flocculated mixture into a solid phase and a liquid phase comprises sieving.

In some embodiments, separating the flocculated mixture into a solid phase and a liquid phase comprises removing a hydrophobic adsorbent. In some embodiments, the removing of a hydrophobic adsorbent comprises centrifugation, such as the use of a disk stack centrifuge, a continuous centrifuge, or a basket centrifuge. In some embodiments, the removing of a hydrophobic adsorbent comprises filtration, including but not limited to using a dead-end filtration system, using ultrafiltration, using a tangential flow filtration system, or using a plate filter. In some embodiments, the removing of a hydrophobic adsorbent comprises use of a press, including but not limited to a screw press, a French press, a belt press, a filter press, a fan press, a finisher press, or a rotary press. In some embodiments, the removing of a hydrophobic adsorbent comprises using gravity settling. In some embodiments, the removing of a hydrophobic adsorbent comprises sieving. In some embodiments, the removing of a hydrophobic adsorbent comprises column filtration. Separation of the flocculated mixture into a solid phase and a liquid phase may yield materials that are useful in various applications, including but not limited to agricultural applications. For example, where the adsorbent is activated carbon, activated charcoal, or activated coal, the liquid phase may comprise soluble proteins and the solid phase may comprise activated carbon, activated charcoal, or activated coal and phenolics, pigments, and/or cellular membranes. Phenolics from the solid phase may be used in human and/or animal nutrition. For example, in some embodiments, the phenolics include carotenoids that may be used in, for example, nutritional supplements. In some embodiments, the phenolics may be used in sunscreen. In some embodiments, the activated carbon, activated charcoal, or activated coal from the solid phase can be reused. In some embodiments, activated carbon, activated charcoal, or activated coal from the solid phase can be applied to plants to, for example, improve moisture retention. In some embodiments, activated carbon, activated charcoal, or activated coal from the solid phase has applications in biofuel technology. For example, in some embodiments, activated carbon, activated charcoal, or activated coal can be used in the production of biochar.

In some embodiments, the liquid phases and/or filtrates for use in the disclosed processes may comprise anti-foaming agents and/or defoaming agents. In some embodiments, the anti-foaming agents and/or defoaming agents are oil defoamers. In some embodiments of oil defoamers, the oil is a mineral oil, a vegetable oil, a white oil, or any oil that is insoluble in a foaming medium except silicone oil. In some embodiments, an oil-based defoamer contains a wax and/or hydrophobic silica. In some embodiments, waxes are selected from ethylene bis-stearamide (EBS), paraffin waxes, ester waxes, and fatty alcohol waxes. In some embodiments, the anti-foaming agents and/or defoaming agents are powder defoamers. In some embodiments, powder defoamers are oil-based defoamers on a particulate carrier, such as silica. In some embodiments, powder defoamers are added to powder products such as cement, plaster, and detergents. In some embodiments, the anti-foaming agents and/or defoaming agents are water-based defoamers. In some embodiments, water-based defoamers comprise one or more oils and/or waxes in a water base, such as mineral oil, vegetable oils, long-chain fatty alcohol, and fatty acid soaps or esters. In some embodiments, the anti-foaming agents and/or defoaming agents are silicon-based defoamers. In some embodiments, silicon-based defoamers are polymers with silicon backbones. In some embodiments, silicon-based defoamers are delivered as an oil- or water-based emulsion. In some embodiments, the silicon compound comprises or consists of a hydrophobic silica dispersed in a silicone oil. In some embodiments, the anti-foaming agents and/or defoaming agents are silicon-based defoamers comprising emulsifiers. In some embodiments, the anti-foaming agents and/or defoaming agents are silicon-based defoamers comprise silicone glycols and/or other modified silicone fluids. In some embodiments, the anti-foaming agents and/or defoaming agents are EO/PO-based defoamers. In some embodiments, the anti-foaming agents and/or defoaming agents are EO/PO-based defoamers comprising polyethylene glycol and/or polypropylene glycol copolymers. In some embodiments, the anti-foaming agents and/or defoaming agents are EO/PO-based defoamers are delivered as oils, water solutions, or water-based emulsions.

Separation of the flocculated mixture into a solid phase and a liquid phase may yield materials that are useful in various applications, including but not limited to agricultural applications. For example, the liquid phase may comprise soluble proteins, excess flocculant (e.g., chitosan), other linked, branched, or linear polysaccharides (including but not limited to ionic, non-ionic, and/or neutral polysaccharides), vitamin B-12, calcium chloride or other divalent ions (e.g., magnesium chloride), RuBisCo, light harvesting complexes/photosystems, soluble proteins, cellular membranes, phenolic compounds, carotenoids, lutein, and/or xanthophylls. For example, the solid phase may comprise chlorophyll, calcium phosphate, cellular membranes, light harvesting complexes/photosystems, and/or chitosan. In some embodiments, this solid phase may be used as, for example, an animal feed or as a biofuel. In some embodiments, this solid phase may contain levulinic acid, which is a potential biofuel precursor. In some embodiments, chlorophyll obtained from the solid phase obtained from the separation of the lysed plant material may be used in, for example, cosmetic applications, as a dye, and/or in human and/or animal nutrition.

In order to stabilize the soluble proteins in the liquid phase, it may be advantageous to perform the steps of the process at low temperatures. Low temperatures may prevent denaturation of the soluble proteins. In some embodiments, separation of the flocculated mixture is performed at no more than about 35° C., 30° C., 25° C., 20° C., 15° C., 10° C., or 5° C. In some embodiments, all steps of the process for making a protein preparation except for the heating step is performed at no more than about 35° C., 30° C., 25° C., 20° C., 15° C., 10° C., or 5° C.

After the separation of the flocculated mixture into a solid phase and a liquid phase, the liquid phase may be filtered to yield a filtrate containing the purified protein. Methods of filtration are well known in the art, and may be performed by use of surface filters or depth filters, for example, by membrane filtration, column filtration, diafiltration, ultrafiltration, tangential flow filtration, etc. In some embodiments, filtration of the liquid phase of the flocculated mixture is performed with a membrane filter. In some embodiments, filtration of the liquid phase of the flocculated mixture is performed with a 5.0 μm, 4.0 μm, 3.0 μm, 2.0 μm, 1.0 μm, 0.7 μm, 0.5 μm, 0.22 μm membrane filter. In some embodiments, filtration of the liquid phase of the flocculated mixture is by surface or depth filtration with diatomaceous earth. In some embodiments, filtration of the liquid phase of the flocculated mixture is performed by surface or depth filtration with silt. In some embodiments, filtration of the liquid phase of the flocculated mixture is performed by surface or depth filtration with activated carbon. In some embodiments, filtration of the liquid phase of the flocculated mixture is performed with up to about 10%, 8%, 6%, 4%, 2%, or 1% activated carbon. In some embodiments, filtration of the liquid phase of the flocculated mixture comprises multiple steps or modes of filtration. For example, filtration may be performed with a membrane filter and an activated carbon bed. In some embodiments, filtering is performed with a 0.2 μm membrane filter and the proteinaceous liquid is exposed to about 2% of activated carbon. In some embodiments, the filtrate is further filtered through membrane filters, e.g., through a 5.0 μm, 4.0 μm, 3.0 μm, 2.0 μm, 1.0 μm, 0.7 μm, 0.5 μm, or 0.2 μm membrane filter.

In some embodiments, small solids and/or microorganisms may be removed from liquid phases and/or filtrates. In some embodiments, small solids and/or microorganisms may be removed from liquid phases and/or filtrates by microfiltration, such as by using a one-pass dead end microfiltration system or a tangential flow filtration system.

In some embodiments, liquid phases and/or filtrates may be sterilized. In some embodiments, liquid phases and/or filtrates are sterilized by microfiltration, such as by using a one-pass dead end microfiltration system or a tangential flow filtration system. In some embodiments, liquid phases and/or filtrates are sterilized by ultraviolet (UV) irradiation. In some embodiments, liquid phases and/or filtrates are sterilized by gamma irradiation. In some embodiments, liquid phases and/or filtrates are sterilized by pasteurization, such as by high pressure pasteurization or high-temperature, short-time pasteurization.

The filtrate comprising the protein preparation may be further concentrated. Methods known in the art to concentrate solutes may be used. In some embodiments, concentrating the filtrate may be performed by ultrafiltration through a filter with as suitable cut-off filter. In some embodiments, concentrating the filtrate may be performed by ultrafiltration through polyethersulfone, polypropylene, polyvinylidene fluoride, polyacrylonitrile, cellulose acetate, or polysulfone. In some embodiments, concentrating the filtrate may be performed by evaporation. concentrating the filtrate may be performed by reverse osmosis. Sizes of cut-off filter may be optimized depending on the protein of interest. In some embodiments, ultrafiltration is performed using a filter with a cut-off of no more than about 200 kDa, 150 kDa, 100 kDa, 75 kDa, 50 kDa, 25 kDa, 10 kDa, or 5 kDa.

In some embodiments, liquid phases and/or filtrates may be dialyzed. In some embodiments, dialysis may be performed using ultrafiltration. In some embodiments, dialysis may be performed using ultrafiltration through polyethersulfone, polypropylene, polyvinylidene fluoride, polyacrylonitrile, cellulose acetate, or polysulfone. In some embodiments, dialysis may be performed using reverse osmosis.

In some embodiments, liquid phases and/or filtrates may be dried. In some embodiments, drying may be accomplished using a spray dryer, a freeze dryer, drum drying, film drying, bed drying, a flash dryer, or a rotary dryer.

The process disclosed herein enables the preparation of a high yield of purified protein. The process disclosed herein may be used to prepare a high purity preparation of the purified protein. Advantageously, the steps disclosed herein may produce a yield of at least about 5%, 10%, 15%, 20%, 25%, 30%, 35%, 40%, 50%, 60%, 70%, 80%, 90%, or 99% of the amount of soluble protein in the liquid phase after lysis of the plant material. In some embodiments, the purity of the protein preparation is at least about 30%, 40%, 50%, 60%, 70%, 80%, 90%, or 95%.

The process disclosed herein may be used to extract appreciable levels of protein from plant material. RuBisCo is an enzyme found in the chloroplast of photosynthetic organisms, which is used to catalyze the first major step of carbon fixation. Up to about 50% of the total protein found in green plant material may consist of RuBisCo, making it the most abundant protein in leaves. In some embodiments, the protein preparation is RuBisCo. In some embodiments, the plant material is from duckweed, algae, beetroot, spinach beet, chard, sugar beet, sea beet, Mangel beet, soy, or tobacco.

Another aspect of the present disclosure relates to a product made by the processes disclosed herein.

Yet another aspect of the present disclosure relates to a food comprising a purified protein preparation from a plant material. Advantageously, the food comprising the protein preparation may contain no more than 30%, 40%, 50%, 60%, 70%, 80%, 90%, or 95% impurities. In some embodiments, the food comprising the protein preparation comprises RuBisCo. In some embodiments, the protein is prepared from plant material from Lemna. In some embodiments, the protein is prepared from plant material from Lemnoideae.

The ability of proteins to form gels and stable foams is important in the production of a variety of foods. As used herein, foams refer to structures formed by trapping pockets of gas in a liquid or solid. Proteins in foams contribute to the foam's ability to form small air cells and stability in holding the structure. Foams with a uniform distribution of small air bubbles impart body, smoothness and lightness to the food. The ability of a protein preparation to form a foam is related to its purity, and a purity of at least about 80% may be needed to form a stable foam. As used herein, gels are soft solids comprising a high amount of an aqueous phase. Protein gels may comprise a three-dimensional network of protein fibers with a continuous liquid phase throughout the matrix. Proteins with higher gelling capacity require less protein to form a gel. The processes disclosed herein may be used to prepare protein preparations with advantageously high purity, foaming capacity, foam stability, and gelling capacity that is suitable for use in food products.

The disclosure is further illustrated by means of the following non-limiting examples.

### EXAMPLES

The soluble protein and freeze-dried protein preparations made by the following processes detailed in Examples 1-4 and Comparative Example 1 were characterized. The concentration of soluble protein in solution prior to freeze-drying was measured by a Pierce 660 nm Protein Assay (Thermo-Scientific Inc.). The purity of the protein was measured by the Dumas method. Foams were produced from each of the soluble preparations, and the foaming characteristics were measured. Foaming capacity (FC) was calculated as:

FC=(Volume after foaming−Volume before foaming)/Volume before foaming×100%.

Foaming stability at a time interval t after foaming was calculated as:

Foaming Stability=Residual Foam Volume at time t/Initial Foam Volume×100%.

### Example 1

One kg of fresh Lemna minor was macerated in a Vitamix Blender (Vitamix Corp, Cleveland, Ohio) in a ratio of 1:1 with a sodium carbonate buffer containing 0.3% w/v sodium bisulfite. The extraction was performed for 3 minutes at medium speed setting maintaining the temperature at less than 30° C. Subsequently, the macerated biomass was filtered by using a nylon straining bag (Natural Home Brands, Sun Valley, Calif.) with a fine mesh to separate the fibrous high solids cake from the liquid juice containing the soluble protein. The filtered homogenate was then centrifuged for 10 minutes at a speed/force of 4000 g (Allegra X15R, SX4750 rotor; Beckman Coulter, Inc., Pasadena, Calif.). The pellet was discarded, and the supernatant was collected separately. The solution was heated to a temperature of 50° C. in a water bath that was set at a temperature of 55° C. and was cooled rapidly to a temperature less than 15° C. after reaching the target temperature. Following the rapid cooling of the protein solution, 2% v/v of activated chitosan and 4% w/v of activated carbon (Cabot Norit Americas Inc, Marshall, Tex.) is added to the liquid juice. The solution was subsequently stirred for 5 minutes after which the solution was centrifuged for 10 minutes at a speed/force of 5000 g (Allegra X15R, SX4750 rotor; Beckman Coulter, Inc., Pasadena, Calif.). The green pellet in the centrifuge bottle was discarded, and the clear yellow supernatant was microfiltered using a 0.7 μm Glass Microfiber membrane (Whatman 1825-047 Glass Microfiber Binder Free Filter, 0.7 Micron; Global Life Sciences Solutions USA LLC, Marlborough, Mass.). The filtrate was subsequently exposed to a 0.2 μm polyethersulfone membrane (Polyethersulfone (PES) Membrane Filters, 0.2 Micron; Sterlitech Corporation Inc, Kent, Wash.) to remove the remainder of the undesired particles including bacteria. The obtained pale yellow and deodorized proteinaceous solution was then concentrated using a 70 kDa membrane (MINIKROS® 502-E070-05-N; Spectrum Laboratories, Inc., Rancho Dominguez, Calif.). The concentrated solution obtained was subsequently freeze dried (Harvest Right LLC, Salt Lake City, Utah) and the result was a white, odorless and soluble protein powder.

### Example 2

One kg of fresh Lemna minor was macerated using a Vitamix Blender (Vitamix Corp, Cleveland, Ohio) in a ratio of 1:1 with a potassium phosphate buffer containing 0.3% w/v ascorbic acid. The maceration was performed for a period of 3 minutes at medium speed in order to maintain a temperature of less than 30° C. The lysed biomass was filtered by using a nylon straining bag (Natural Home Brands, Sun Valley, Calif.) with a fine mesh to separate the fibrous high solids cake from the liquid juice containing the soluble protein. The filtered homogenate was then centrifuged for 10 minutes at a speed/force of 4000 g (Allegra X15R, SX4750 rotor; Beckman Coulter, Inc., Pasadena, Calif.). The pellet was discarded, and the supernatant was collected separately. The supernatant was then mixed with 5% v/v of activated chitosan (Chitosan (10-120 cps), fungal origin (9012-76-4); Glentham Life Sciences Ltd., Corsham, Wiltshire, UK) and 10% w/v of activated carbon (Cabot Norit Americas Inc, Marshall, Tex.) for a period of 5 minutes. Subsequently the mixed solution was centrifuged at a speed/force of 5000 g for 10 minutes (Allegra X15R, SX4750 rotor; Beckman Coulter, Inc., Pasadena, Calif.). The obtained pellet was discarded, and the deodorized and decolored supernatant was microfiltered using a 0.2 μm polyethersulfone membrane (Polyethersulfone (PES) Membrane Filters, 0.2 Micron; Sterlitech Corporation Inc, Kent, Wash.). The obtained pale yellow and deodorized proteinaceous solution was then concentrated using a 70 kDa membrane (MINIKROS® 502-E070-05-N; Spectrum Laboratories, Inc., Rancho Dominguez, Calif.). The concentrated solution obtained was subsequently freeze dried (Harvest Right LLC, Salt Lake City, Utah) and the result was a white, odorless and soluble protein powder.

### Example 3

One kg of fresh Lemna minor was macerated using a Vitamix Blender (Vitamix Corp, Cleveland, Ohio) in a ratio of 1:1 with distilled water containing 0.3% w/v of sodium bisulfite and ascorbic acid. The maceration was performed for a period of 3 minutes at medium speed in order to maintain a temperature of less than 30° C. The lysed biomass was filtered by using a nylon straining bag (Natural Home Brands, Sun Valley, Calif.) with a fine mesh to separate the fibrous high solids cake from the liquid juice containing the soluble protein. The filtered homogenate was then centrifuged for 10 minutes at a speed/force of 4000 g. The pellet was discarded, and the supernatant was collected separately. The supernatant was then mixed with a solution containing 30 mM of potassium phosphate and 20 mM of calcium chloride for a period of 5 minutes. Subsequently the mixed solution was centrifuged at a speed/force of 5000 g for 10 minutes (Allegra X15R, SX4750 rotor; Beckman Coulter, Inc., Pasadena, Calif.). The obtained pellet was discarded. 5% w/v of activated carbon (Cabot Norit Americas Inc, Marshall, Tex.) was added to the supernatant, and the solution was stirred for 5 minutes. Subsequently, the mixed solution containing the activated carbon was microfiltered using a 0.2 μm polyethersulfone membrane filter (Polyethersulfone (PES) Membrane Filters, 0.2 Micron; Sterlitech Corporation Inc, Kent, Wash.) in order to remove the activated carbon that had adsorbed the remaining chlorophyll, polyphenol and other unwanted taste/color/odor impacting particles. The obtained pale yellow and deodorized proteinaceous solution was then concentrated using a 100 kDa membrane (Hollow Fiber Cartridge, 100,000 NMWC, 850 cm2; GE Healthcare Bio-Sciences Corp, Westborough, Mass.). The concentrated solution obtained was subsequently freeze dried and the result was a white, odorless and soluble protein powder.

### Example 4

One kg of fresh Lemna minor was macerated using a Vitamix Blender (Vitamix Corp, Cleveland, Ohio) in a ratio of 1:1 with distilled water containing 0.5% w/v of sodium bisulfite. The maceration was performed for a period of 3 minutes at medium speed in order to maintain a temperature of less than 30° C. The lysed biomass was filtered by using a nylon straining bag (Natural Home Brands, Sun Valley, Calif.) with a fine mesh to separate the fibrous high solids cake from the liquid juice containing the soluble protein. The filtered homogenate was then centrifuged for 10 minutes at a speed/force of 4000 g (Allegra X15R, SX4750 rotor; Beckman Coulter, Inc., Pasadena, Calif.). The pellet was discarded, and the supernatant was collected separately. The supernatant was then mixed with a solution containing 30 mM of potassium phosphate and 20 mM of calcium chloride for a period of 5 minutes. Subsequently the mixed solution was centrifuged at a speed/force of 5000 g for 10 minutes (Allegra X15R, SX4750 rotor; Beckman Coulter, Inc., Pasadena, Calif.). The obtained pellet was discarded. 2% w/v of activated chitosan (Chitosan (10-120 cps), fungal origin (9012-76-4); Glentham Life Sciences Ltd., Corsham, Wiltshire, UK) and 4% of activated carbon (Cabot Norit Americas Inc, Marshall, Tex.) were added to the supernatant, and the solution was stirred for 5 minutes. Subsequently the mixed solution was centrifuged at a speed/force of 5000 g for 10 minutes (Allegra X15R, SX4750 rotor; Beckman Coulter, Inc., Pasadena, Calif.). The obtained pellet was discarded, and the deodorized and decolored supernatant was microfiltered using a 0.7 μm polyethersulfone membrane (Whatman 1825-047 Glass Microfiber Binder Free Filter, 0.7 Micron; Global Life Sciences Solutions USA LLC, Marlborough, Mass.). The filtrate was then further microfiltered using a 0.2 μm polyethersulfone membrane (Polyethersulfone (PES) Membrane Filters, 0.2 Micron; Sterlitech Corporation Inc, Kent, Wash.). The obtained pale yellow and deodorized proteinaceous solution was then concentrated using a 70 kDa membrane (MINIKROS® 502-E070-05-N; Spectrum Laboratories, Inc., Rancho Dominguez, Calif.). The concentrated solution obtained was subsequently freeze dried (Harvest Right LLC, Salt Lake City, Utah) and the result was a white, odorless and soluble protein powder.

Results from Examples 1-4

The average purity of the protein preparations prepared by the methods of Examples 1-4 was ˜84.3% and the concentration of soluble protein after ultrafiltration was 1,316 μg/mL. The foaming capacity achieved was 195% and maintained a 92% stability after 1 hour. Gelation properties of the freeze-dried material were validated, and only 2% w/v of freeze-dried material was needed to be added in order to form a gel.

### Comparative Example 1

Lemna leaf proteins were extracted as described in WO2011/0778671 A1 (van de Velde et al.) with some modifications.

One kg of fresh Lemna was washed and macerated using a Vitamix Blender at a ratio of 2:1 with 0.3% w/v sodium bisulfite. The homogenate was sieved through a cheese cloth prior to heating up to 60° C. The filtrate was kept at 60° C. for 5 minutes and then cooled down to 10° C. Following the heat treatment, the suspension was centrifuged for 5 minutes at 5200 g. Next, activated carbon was added to the supernatant in an amount of 5% w/w. Following the addition of the activated carbon, the suspension was stirred for 5 minutes before the supernatant was removed by decanting.

The supernatant obtained was subjected to two microfiltration steps. First, the supernatant was passed over a microfilter having a pore size of 0.7 μm (Whatman 1825-047 Glass Microfiber Binder Free Filter, 0.7 Micron; Global Life Sciences Solutions USA LLC, Marlborough, Mass.). Subsequently, the filtrate was passed over a microfilter having a pore size 0.2 μm (Polyethersulfone (PES) Membrane Filters, 0.2 Micron; Sterlitech Corporation Inc, Kent, Wash.). The filtrate was then freeze dried and the result was a whitish and odorless powder.

Results from Comparative Example 1

The purity of the protein was approximately 34.1% per unit of dry matter and the concentration of soluble protein prior to freeze-drying was 520 μg/mL. The foaming properties of the freeze-dried material showed a total foaming strength of 92% with a stability of 62% after 1 hour. Gelation properties of the freeze-dried material were validated, and at least 7% w/v of freeze-dried material was needed to be added in order to form a gel.

TABLE 1

| | Purity (% per |
|---|---|
| Sample | unit of dry matter) |
| Example 1 | 88.2 |
| Example 2 | 85.2 |
| Example 3 | 82.1 |
| Example 4 | 78.9 |
| Comparative | 34.1 |
| Example 1 | |

### Example 5

This example investigated the removal of chlorophyll using calcium chloride to coagulate chlorophyll-protein complexes.

2 kg of biomass was lysed (Vitamix Corp, Cleveland, Ohio) with an extraction buffer comprising 2% metabisulfite, 0.1 M NaCl. Four fractions were then made from the filtrate, all having the volume of 375 mL. All fractions were mixed at speed 4 and centrifugation was for all fractions and steps set at 5200 g and 5 minutes. For filtration, a Buchner funnel was used with a 0.45 □m cutoff filter sheet (Polyethersulfone (PES) Membrane Filters, 0.45 Micron; Sterlitech Corporation Inc, Kent, Wash.) coated in DE (Dicalite Management Group Inc, Bala Cynwyd, Pa.).

Fraction 1: 3.75 g of phosphate buffer was added to get a concentration of 10 mM in the fraction. The fraction was then run through the heat bath step set to 68° C. after which 15 g activated carbon (Cabot Norit Americas Inc, Marshall, Tex.) was added and mixed for 25 minutes. Following that 15 g of 3% chitosan (Chitosan (10-120 cps), fungal origin (9012-76-4); Glentham Life Sciences Ltd., Corsham, Wiltshire, UK) was added and mixed for an additional 5 minutes. The solution was then centrifuged and then filtered.

Fraction 2: 3.75 g of phosphate buffer was added to get a concentration of 10 mM in the fraction. 15 g of activated carbon (Cabot Norit Americas Inc, Marshall, Tex.) was then added to the fraction and mixed for 15 minutes. Following that, 15 g of 3% chitosan (Chitosan (10-120 cps), fungal origin (9012-76-4); Glentham Life Sciences Ltd., Corsham, Wiltshire, UK) was added and mixed for an additional 5 minutes. The solution was then centrifuged and then filtered.

Fraction 3: 3.75 g of phosphate buffer and 2.81 g of calcium chloride solution was added to get a concentration of 10 mM and 7.5 mM respectively in the fraction. 15 g of activated carbon (Cabot Norit Americas Inc, Marshall, Tex.) was then added to the fraction and mixed for 15 minutes. Following that, 15 g of 3% chitosan (Chitosan (10-120 cps), fungal origin (9012-76-4); Glentham Life Sciences Ltd., Corsham, Wiltshire, UK) was added and mixed for an additional 5 minutes. The solution was then centrifuged and then filtered.

Fraction 4: 7.5 g of phosphate buffer and 5.63 g of calcium chloride solution was added to get a concentration of 20 mM and 15 mM respectively in the fraction. 15 g of activated carbon (Cabot Norit Americas Inc, Marshall, Tex.) was then added to the fraction and mixed for 15 minutes. Following that, 15 g of 3% chitosan (Chitosan (10-120 cps), fungal origin (9012-76-4); Glentham Life Sciences Ltd., Corsham, Wiltshire, UK) was added and mixed for an additional 5 minutes. The solution was then centrifuged and then filtered.

TABLE 2
Schematic Variable Table

| | | | Calcium |
|---|---|---|---|
| | | Phosphate | chloride |
| | Heat | concentration | concentration |
| Fraction | Bath | in mM | in mM |
| 1 | Yes | 10 | 0 |
| 2 | No | 10 | 0 |
| 3 | No | 10 | 7.5 |
| 4 | No | 20 | 15 |

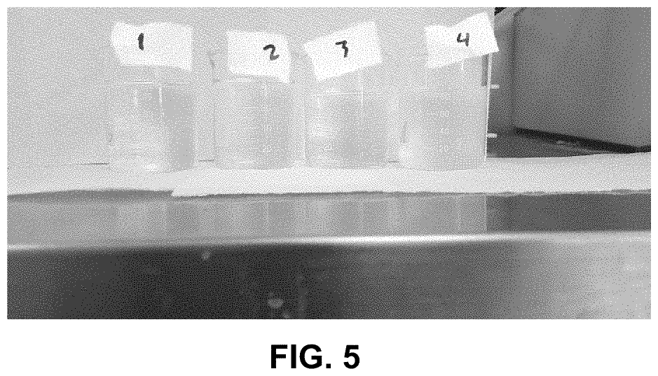

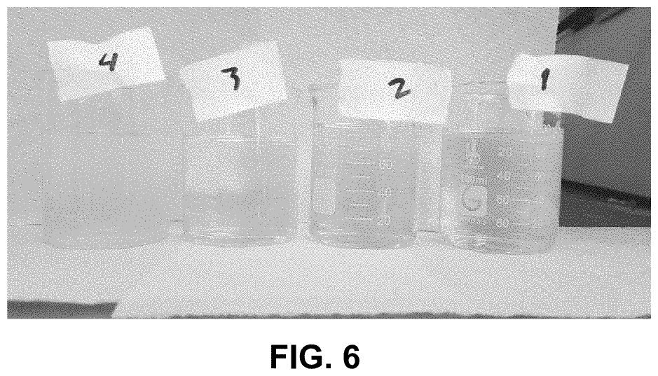

FIG. 5 depicts Fractions 1, 2, 3, and 4 after microfiltration. FIG. 6 depicts Fractions, 4, 3, 2, and 1 after microfiltration. As depicted in FIG. 5 and FIG. 6, coagulation with calcium chloride does not show a significant difference in the removal of chlorophyll relative to the control Fraction (Fraction 2). As also depicted in FIG. 5 and FIG. 6, the use of two different concentrations of calcium chloride in Fractions 3 and 4 did not result in a significant difference in chlorophyll removal.

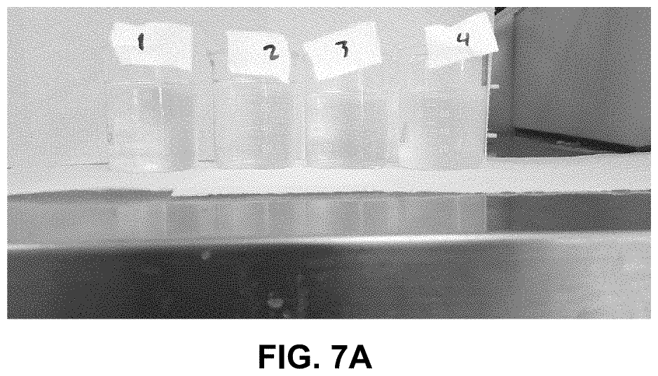

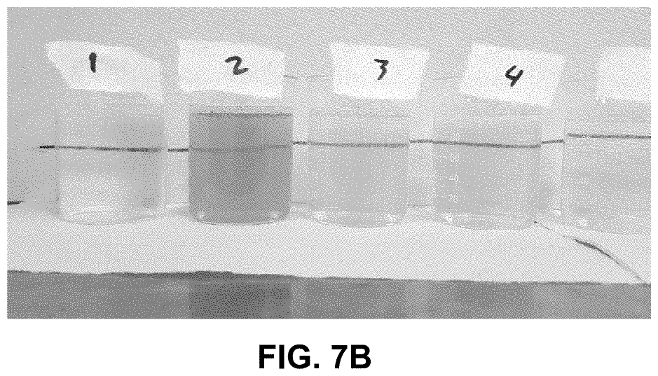

A further experiment examined the point at which EDTA is added. For a first set of Fractions, the filtrate obtained after solid/liquid separation using a basket centrifuge was treated with phosphate (comprising potassium phosphate dibasic and potassium phosphate monobasic) but without EDTA. FIG. 7A depicts these Fractions. For a second set of Fractions, the lysate obtained before solid/liquid separation was treated with phosphate (comprising potassium phosphate dibasic and potassium phosphate monobasic) and with EDTA. FIG. 7B depicts these Fractions. As shown in FIG. 7A and FIG. 7B, Fraction 2 from the second set of Fractions has a dramatically different color compared to Fraction 2 from the first set of fractions. Without wishing to be bound by theory, it is believed that these results suggest that adding EDTA to the filtrate leads to a greater removal of color compared to adding EDTA to the lysate.

### Example 6

This example investigated the use of calcium chloride to coagulate chlorophyll and/or chloroplast membranes as an alternative to using a heat bath.

Biomass was lysed in extraction buffer containing 0.1 M NaCl and 2% metabisulfite (without EDTA). Calcium chloride and phosphate (comprising potassium phosphate dibasic and potassium phosphate monobasic) were added to 375 mL of post-basket centrifugation (Rousselet-Robatel Model RA20VxR Vertical Basket Centrifuge; Robatel Inc, Pittsfield, Mass.) filtrate in the amounts detailed in Table 4 below.

TABLE 4
Schematic Variable Overview

| | Calcium | | | pH after |
|---|---|---|---|---|
| | chloride | Phosphate | pH during | basket |
| | in mM | in mM | lysing | centrifugation |
| Fraction 1 | 0 | 100 | 7 | 7 |
| Fraction 2 | 7.5 | 10 | 7 | 7 |
| Fraction 3 | 30 | 40 | 7 | 7 |
| Fraction 4 | 75 | 100 | 7 | 7 |
| Fraction 5 | 75 | 0 | 7 | 7 |
| Fraction 6 | 75 | 100 | 7 | 7.5 |
| Fraction 7 | 75 | 100 | 7.5 | 7.5 |

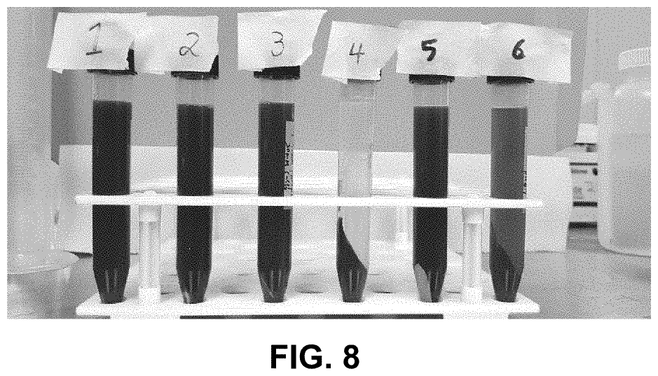

The filtrate was stirred for 15 minutes at room temperature. After calcium chloride treatment, a 13 mL fraction was taken and spun down on a tabletop centrifuge (Horizon Model 614B Centrifuge; Drucker Diagnostics LLC, Port Matilda, Pa.). The color of the supernatant and the weight of the pellet fraction was measured. FIG. 8 depicts samples of the fractions after calcium chloride and phosphate addition and approximately five minutes of benchtop centrifugation. As depicted in FIG. 8, Fraction 4 demonstrated superior chlorophyll removal. As also depicted in FIG. 8, Fraction 5 contained a white pellet (content unknown) while the supernatant remained relatively chlorophyll-filled. Without wishing to be bound by theory, the results may suggest that phosphate is necessary for calcium chloride to remove chlorophyll effectively at a concentration of 75 mM. As also depicted in FIG. 8, Fraction 6, which was lysed at pH 7.5, demonstrated relatively lower chlorophyll removal relative to Fraction 4. Without wishing to be bound by theory, the results may suggest that calcium chloride is less effective at a higher pH.

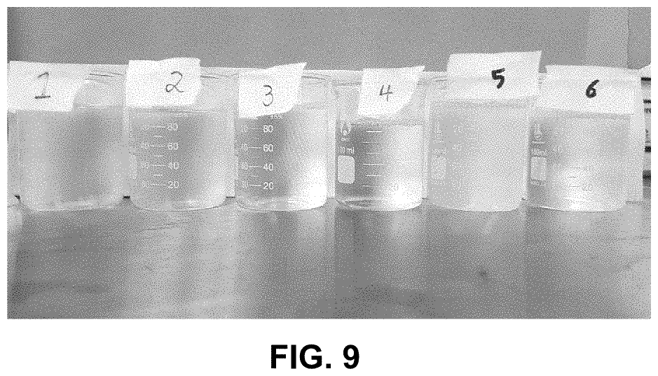

The remainder of the lysate was then treated with activated carbon Cabot Norit Americas Inc, Marshall, Tex.) (15 minutes) and chitosan (Chitosan (10-120 cps), fungal origin (9012-76-4); Glentham Life Sciences Ltd., Corsham, Wiltshire, UK) (5 minutes) per standard procedure. The activated carbon-chitosan was spun down, and the supernatant was further filtered through 2 coffee filters. The color of the supernatant was noted. FIG. 9 depicts samples of Fractions 1-6 after removal of activated carbon and chitosan. As depicted in FIG. 9, chitosan and activated carbon function properly after pre-treatment with calcium chloride and phosphate. As also depicted in FIG. 9, the color removal by activated carbon and chitosan were most effective on Fraction 6, which contained 75 mM calcium chloride and 100 mM phosphate.

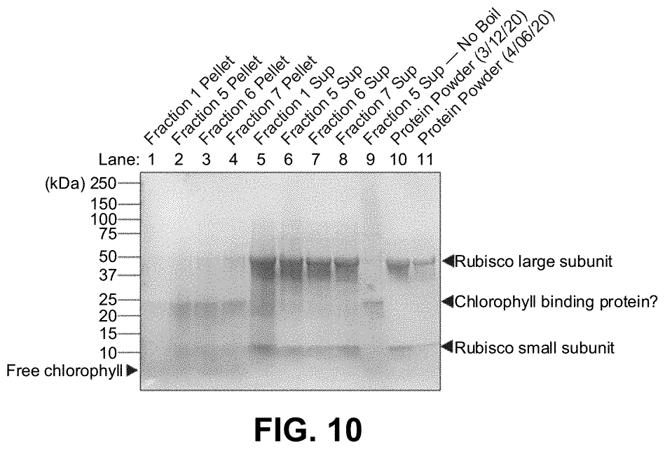

SDS-PAGE Coomassie staining analysis was performed to visualize and determine Rubisco protein levels in Fractions 1, 5, 6, and 7. FIG. 10 depicts results from the SDS-PAGE Coomassie staining analysis. FIG. 10 depicts SDS-PAGE gel (Bio-Rad Laboratories, INC, Hercules, Calif.) results from the pellet and supernatant (“Sup”) after benchtop centrifugation for Fractions 5-7. As depicted in FIG. 10, lane 9 shows that chlorophyll is still attached to a protein around 25 kDa, while lane 6 shows that chlorophyll is detached. Without wishing to be bound by theory, these results may suggest that the protein that is attached to chlorophyll has a (subunit) size of approximately 25 kDa. As also depicted in FIG. 10, Rubisco is for the large majority in the Sup of Fractions 5, 6, and 7, while the 25 kDa-chlorophyll-binding protein is for the most part in the pellet. Without wishing to be bound by theory, these results may suggest that calcium chloride with phosphate selectively precipitates the chlorophyll-binding protein and leaves Rubisco in solution.

Without wishing to be bound by theory, it is believed that the results indicate that calcium chloride efficiently removed chlorophyll and cellular membranes from green filtrate post-basket centrifugation, that the bulk of Rubisco remained in the supernatant, that calcium chloride-induced precipitation appears to occur immediately, and that calcium chloride removes 25 kDa-chlorophyll-binding protein.

### Example 7

This example investigated the effect of 0.5% detergent on protein recovery from filtering the lysate and the effect of detergent on downstream process steps.

4 kg of biomass was lysed (Vitamix Corp, Cleveland, Ohio) with a buffer containing 0.1 M NaCl, 0.1M phosphate (comprising potassium phosphate dibasic and potassium phosphate monobasic), and 2% metabisulfite. After mixing 2 L of the lysate with 200 ml 10% Chaps solution (Biovision Inc, Milpitas, Calif.) to get a 0.5% concentration in the lysate. This was then mixed for ˜10 minutes and spun out in a basket centrifuge (Rousselet-Robatel Model RA20VxR Vertical Basket Centrifuge; Robatel Inc, Pittsfield, Mass.). To the remaining lysate, 200 mL water was added to correct for the dilution factor.

Fractions of 375 ml of filtrate for both fractions were taken and 28.1 ml of 1M calcium chloride buffer was added to get 75 mM concentration. The mixture was spun out and the supernatants were compared. The control supernatant was taken and 5% of normal chitosan (Chitosan (10-120 cps), fungal origin (9012-76-4); Glentham Life Sciences Ltd., Corsham, Wiltshire, UK) amount was added (0.75 g to 375 ml). The solution was mixed for 5 minutes after which a small 13 mL sample was taken and spun down on the benchtop centrifuge (Horizon Model 614B Centrifuge; Drucker Diagnostics LLC, Port Matilda, Pa.). The pH of the supernatant was then raised to 7.2 to check for remaining chitosan and sufficient colour removal. Another 0.75 g was added to the original solution to get a total of 10% of normal chitosan (1.5 g in 375 mL). This was repeated until colour removal was sufficient or excess chitosan was observed. The chitosan % at which this was observed was 10%. 25% of AC and 10% of chitosan were added to both fractions and spun down with the centrifuge.

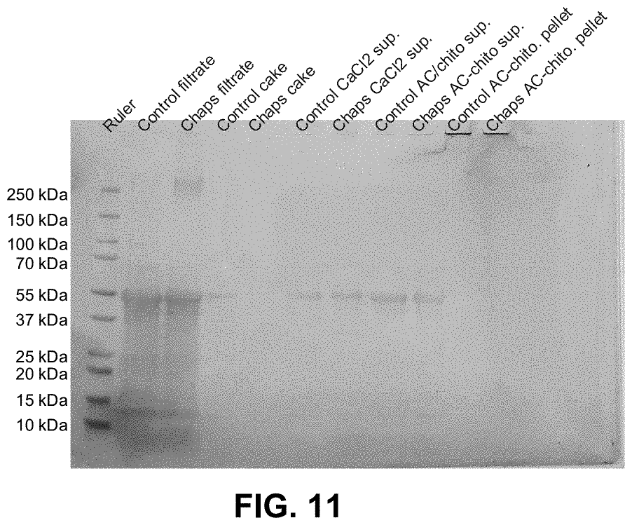

FIG. 11 depicts an SDS-PAGE gel (Bio-Rad Laboratories, INC, Hercules, Calif.) on various samples. As depicted in FIG. 11, the detergent does not significantly increase the Rubisco in the filtrate, and there does not appear to be Rubisco in the activated carbon-chitosan pellet in either fraction.

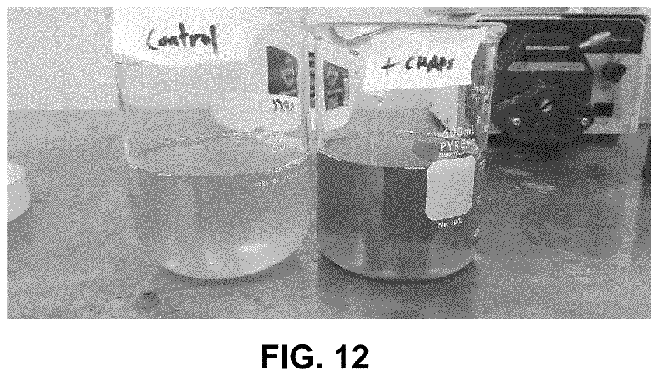

FIG. 12 depicts the fractions after removal of activated carbon and chitosan. As depicted in FIG. 12, treatment with Chaps releases more polyphenols and/or polyphenol oxidase (PPO) during lysing.

Without wishing to be bound by theory, these results may suggest that treatment with Chaps does not significantly release more Rubisco during lysing but that treatment with Chaps does release a greater amount of polyphenols and/or polyphenol oxidase (PPO) during lysing. Also without wishing to be bound by theory, these results may also suggest that 25% activated carbon and 5% chitosan did not significantly remove color in either fraction.

### Example 8

This example related to a calcium chloride baseline run, resuspension of a cake with 0.1% CHAPS, and a wash of second cake with 0.1% CHAPS.

2 kg of biomass was lysed (Vitamix Corp, Cleveland, Ohio) with an extraction buffer comprising 0.2M NaCl, 0.1M PO₄ pH 7.7, 2% metabisulfite, (recipe per kg: 20 ml 5M NaCl, 115 ml 1M NaOH, 115 ml H2O, 50 ml 1M PO4 buffer pH 7.6, 200 g ice). The 2 kg was eventually split into three fractions. For Fraction 1, 75 mM calcium chloride was added after lysing with Vitamix, and mixed by hand for 10 minutes, while closely watching the pH. It was then spun out with a basket centrifuge (Rousselet-Robatel Model RA20VxR Vertical Basket Centrifuge; Robatel Inc, Pittsfield, Mass.), and the fraction was collected as the filtrate. The preparation of Fraction 2 comprised resuspending the basket centrifugation cake in 2 L of resuspension buffer (70 mM PO₄ pH7.2, 0.1M NaCl, 0.1% CHAPS). The slurry was then Vitamix blended for 1 minute, and the filtrate (Fraction 2) and cake were separated by basket centrifugation. Very similar to Fraction 2, Fraction 3 was prepared by resuspending the basket centrifugation cake in 1 L of resuspension buffer. The slurry was mixed by hand, and allowed to sit for 10 minutes prior to basket centrifugation. 50 g of activated carbon was added and mixed for 15 minutes followed by 20 g of 3% chitosan (Chitosan (10-120 cps), fungal origin (9012-76-4); Glentham Life Sciences Ltd., Corsham, Wiltshire, UK) mixed for an additional 5 minutes per fraction. The solution was then spun down and microfiltered through a 0.2 μm filter (Polyethersulfone (PES) Membrane Filters, 0.2 Micron; Sterlitech Corporation Inc, Kent, Wash.). Lastly, the solution was concentrated down with the 50 kDa ultrafiltration (MINIKROS® 502-E050-05-N; Spectrum Laboratories, Inc., Rancho Dominguez, Calif.), and diafiltered until salinity was below 0.1 ppt (˜10 L dH₂O). The last step was the 10 kDa ultrafiltration (MINIKROS® 502-E010-05-N; Spectrum Laboratories, Inc., Rancho Dominguez, Calif.) after which it was freeze-dried. The parameters used during the process of control/cake resuspension/second wash are provided in Table 5.

TABLE 5
Process Parameters

| | | Adjusted | Volume |
|---|---|---|---|
| Step | pH | to pH | in L |
| Lysate | 7.1 for all | 7.7 for all | 3.3/3.0/2.3 |
| Lysate with | 6.3 | 6.8 | — |
| calcium chloride | | | |
| Filtrate | 6.6/6.8/7.1 | 7.3 for all | 2.5/2/1.7 |
| Filtrate with | 6.8 for all | — | — |
| chitosan | | | |

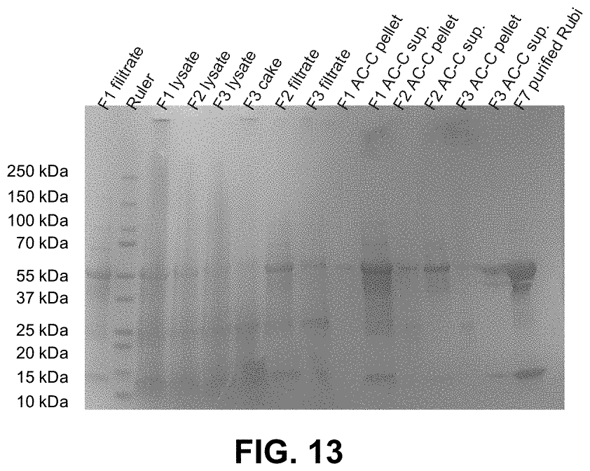

After basket centrifugation, Fraction 2 and Fraction 3 had a deeper green color relative to Fraction 1. However, after microfiltration, Fraction 3 was almost colorless, and Fraction 2 was lighter in color than Fraction 1. FIG. 13 depicts an SDS-PAGE gel for various samples, wherein “F1” refers to “Fraction 1,” “F2” refers to “Fraction 2,” “F3” refers to “Fraction 3,” “AC-C” refers to “activated carbon-chitosan,” “sup” refers to “supernatant,” and “Rubi” refers to “Rubisco.” A 94.25% yield based on total soluble protein content in the biomass of soluble crude protein was observed.

### Example 9

This example investigated treatment with calcium chloride, increased phosphate, and 0.25% CHAPS detergent.

A total of 6 kg of biomass was processed (Vitamix Corp, Cleveland, Ohio). The 6 kg was divided into three 2 kg batches to run three experimental fractions. Fraction 1 was lysed in extraction buffer comprising 0.2M NaCl, 0.1M phosphate (comprising potassium dibasic phosphate and potassium monobasic phosphate), pH 7.7, and 2% metabisulfite, (recipe per kg: 20 ml 5M NaCl, 120 ml 1M NaOH, 110 ml H₂O, 50 ml 1M phosphate buffer pH 7.6, 200 g ice).

Lysis was performed via Vitamixing for 3 minutes at Power 5. Fraction 2 was lysed in the same fashion as Fraction 1; however, the extraction buffer contained more phosphate (recipe per kg: 20 ml 5M NaCl, 120 ml 1M NaOH, 77 ml H₂O, 83 ml 1M phosphate buffer pH 7.6, 200 g ice). Fraction 3 was lysed in the same phosphate buffer as Fraction 2 except a final concentration of 0.25% CHAPS detergent (Biovision Inc, Milpitas, Calif. was added. Lysis occurred in the Vitamix blender using the same protocol as Fractions 1 and 2 (some foaming was observed).

The pH of the filtrates was adjusted to pH 7.3 with 1M NaOH, and never allowed to go below pH 6.8. 75 mM calcium chloride was added after lysing and mixed by hand for 10 minutes while closely watching the pH. It was then spun out with a basket centrifuge (Rousselet-Robatel Model RA20VxR Vertical Basket Centrifuge; Robatel Inc, Pittsfield, Mass.) to collect filtrate. Preparation of Fraction 2 consisted of resuspending the basket centrifuge cake in 2 L of resuspension buffer (70 mM PO4 pH7.2, 0.1M NaCl, 0.1% CHAPS). The slurry was then Vitamix blended for 1 minute, and the filtrate (Fraction 2) and cake were separated by basket centrifugation. Very similar to Fraction 2, Fraction 3 was prepared by resuspending the basket centrifugation cake in 1 L of resuspension buffer. The slurry was mixed by hand and allowed to sit for 10 minutes prior to basket centrifugation. 50 g of activated carbon (Cabot Norit Americas Inc, Marshall, Tex.) was added and mixed for 15 minutes followed by 20 g of 3% chitosan (Chitosan (10-120 cps), fungal origin (9012-76-4); Glentham Life Sciences Ltd., Corsham, Wiltshire, UK) mixed for an additional 5 minutes per fraction. The solution was then spun down and microfiltered through a 0.2 □m filter (Polyethersulfone (PES) Membrane Filters, 0.2 Micron; Sterlitech Corporation Inc, Kent, Wash.). Lastly, the solution was concentrated down with the 50 kDa ultrafiltration (MINIKROS® S02-E050-05-N; Spectrum Laboratories, Inc., Rancho Dominguez, Calif.), and diafiltered until salinity was below 0.1 ppt (˜10 L dH2O). The last step was the 10 kDa ultrafiltration (MINIKROS® S02-E010-05-N; Spectrum Laboratories, Inc., Rancho Dominguez, Calif.) after which it was freeze-dried (Harvest Right LLC, Salt Lake City, Utah). Parameters used during the process of control/Fraction 2/Fraction 3 are provided in Table 6.

TABLE 6
Process Parameters

| | | Adjusted | Volume |
|---|---|---|---|
| Step | pH | to pH | in L |
| Lysate | 7.1 for all | 7.7 for all | 3.4/3.4/4.8 |
| Lysate with | 6.3 | 6.8 | — |
| calcium chloride | | | |
| Filtrate | 6.6/6.8/7.1 | 7.3 for all | 2.8/2.5/2.6 |
| Filtrate with | 6.8 for all | — | — |
| chitosan | | | |

Fraction 2 was slightly darker than fraction 1 after basket centrifugation, fraction 3 was significantly darker green after basket centrifugation. Both fraction 2 and 3 were more turbid relative to fraction 1 after ultrafiltration. The volume of Fraction 3 was 4.8 L; however, it was treated as 3.4 L due to the obviously increased amount of air that inflated the volume. The volumes of the filtrates were all reduced to 2.5 L before processing. Accordingly, 89% of Fraction 1, 100% of Fraction 2, and 96% of Fraction 3 were actually used.

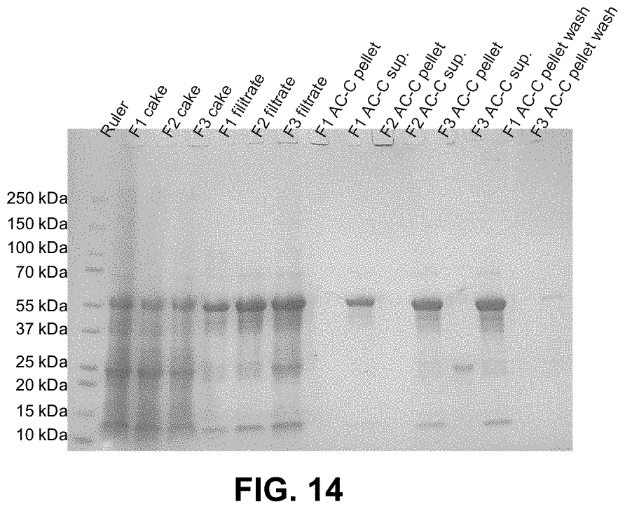

FIG. 14 depicts an SDS-PAGE gel for various samples, wherein “F1” refers to “Fraction 1,” “F2” refers to “Fraction 2,” “F3” refers to “Fraction 3,” “AC-C” refers to “activated carbon-chitosan,” and “sup” refers to “supernatant.” The yields based on total soluble protein content in the biomass were as follows: Control, 80.69%; Fraction 2: 89.43%; Fraction 3: 95.20%.

### Example 10

This example investigated the effect of chitosan concentration on chlorophyll removal without using a heat bath.

2 kg of biomass was lysed (Vitamix Corp, Cleveland, Ohio) with an extraction buffer comprising 2% metabisulfite, 10 mM of EDTA, and 0.1 M NaCl. Five fractions were then made from the filtrate, all having the volume of 375 mL. All fractions were mixed at speed 4 and centrifugation (Allegra X15R, SX4750 rotor; Beckman Coulter, Inc., Pasadena, Calif.) was for all fractions and steps set at 5200 g and 5 minutes. For filtration, a coffee filter was used followed by a Buchner funnel with a 0.45 □m cutoff filter sheet (Polyethersulfone (PES) Membrane Filters, 0.45 Micron; Sterlitech Corporation Inc, Kent, Wash.) coated in DE (Dicalite Management Group Inc, Bala Cynwyd, Pa.).

Fraction 1: 15 g of activated carbon (Cabot Norit Americas Inc, Marshall, Tex.) was then added to the fraction and mixed for 15 minutes. Following that, 15 g of 1% chitosan (Chitosan (10-120 cps), fungal origin (9012-76-4); Glentham Life Sciences Ltd., Corsham, Wiltshire, UK) was added and mixed for an additional 5 minutes. The solution was then centrifuged (Allegra X15R, SX4750 rotor; Beckman Coulter, Inc., Pasadena, Calif.) and then filtered (Polyethersulfone (PES) Membrane Filters, 0.2 Micron; Sterlitech Corporation Inc, Kent, Wash.).

Fraction 2: 15 g of activated carbon was then added to the fraction and mixed for 15 minutes. Following that, 15 g of 2% chitosan was added and mixed for an additional 5 minutes. The solution was then centrifuged and then filtered.

Fraction 3: 15 g of activated carbon was then added to the fraction and mixed for 15 minutes. Following that, 15 g of 3% chitosan was added and mixed for an additional 5 minutes. The solution was then centrifuged and then filtered.

Fraction 4: 15 g of activated carbon was then added to the fraction and mixed for 15 minutes. Following that, 15 g of 4% chitosan was added and mixed for an additional 5 minutes. The solution was then centrifuged and then filtered.

Fraction 5: 15 g of activated carbon was then added to the fraction and mixed for 15 minutes. Following that, 15 g of 5% chitosan was added and mixed for an additional 5 minutes. The solution was then centrifuged and then filtered. However, a mistake was made and the timer was not started after chitosan addition. Therefore, the results for Fraction 5 were not compared with the other Fractions.

Schematic Variable Overview
Range of chitosan: from 1% to 5%
No phosphate buffer
15 min of 100% activated carbon mixing followed by 5 min of chitosan mixture.

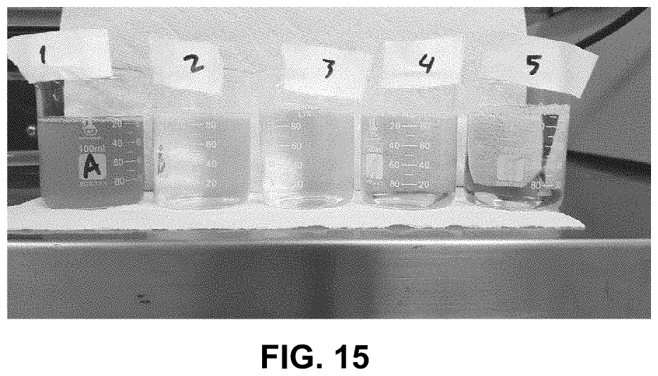

FIG. 15 depicts Fractions 1-5 after microfiltration with the Buchner funnel. As depicted in FIG. 15, the color was removed seemingly equally well in Fractions 2, 3, and 4. Without wishing to be bound by theory, a comparison between Fraction 1 and Fraction 2 suggests that 1% chitosan did not remove chlorophyll as effectively as 2% chitosan. As depicted in FIG. 15, Fraction 5, which received approximately 2-4 minutes of additional exposure to activated carbon and chitosan relative to Fractions 1-4, was clearer than Fractions 1-4.

Without wishing to be bound by theory, these results may suggest that treatment with a 2% solution of chitosan works approximately as well as treatment with a 3% solution of chitosan or a 4% solution of chitosan. Without wishing to be bound by theory, these results may suggest that treatment with, e.g., a 5% solution of chitosan and with extended exposure time to activated carbon and chitosan may also improve chlorophyll removal.

### Example 11

This example investigated the use of activated bentonite clay (CC160 from EP Engineered Clays) to remove colored compounds from post-chitosan lysate in place of, or in conjunction with, activated carbon (Cabot Norit Americas Inc, Marshall, Tex.).

Fresh supernatant (post-chitosan spindown in the extraction process) was obtained, and its pH was increased to 7.0 with NaOH. The optical density was measured by Pierce assay (Pierce™ 660 nm Protein Assay Reagent; Thermo Fisher Scientific, Waltham, Mass.), and the absorbance at 474 nm was measured to determine the starting point for [protein] and the amount of orange discoloration. As a person having ordinary skill in the art would understand, the Pierce 660 nm Protein Assay Reagent can be used to measure total protein concentration. Without wishing to be bound by theory, it is believed that the Pierce 660 nm Protein Assay is based on the binding of a dye-metal complex to protein that causes a shift in the dye's absorption maximum, which is measured at 660 nm. The dye-metal complex is reddish-brown and changes to green upon protein binding, and the color produced in the assay increases in proportion to increasing protein concentrations. To a 100 mL sample was added 0.3% w/v activated carbon (Cabot Norit Americas Inc, Marshall, Tex.). The mixture was stirred for 1 minute before being poured directly onto a Buchner funnel with a 0.45 □ filter Polyethersulfone (PES) Membrane Filters, 0.45 Micron; Sterlitech Corporation Inc, Kent, Wash.), followed by a 0.2 □ filter (Polyethersulfone (PES) Membrane Filters, 0.2 Micron; Sterlitech Corporation Inc, Kent, Wash.), collecting the filtrate in a flask using vacuum. The optical density was measured by Pierce assay, and the absorbance at 474 nm was measured. To a separate 100 mL sample was added 0.3% w/v CC160 clay. The mixture was stirred for 1 minute before being poured directly onto a Buchner funnel with a 0.45 □ filter, followed by a 0.2 □ filter, collecting the filtrate in a flask using vacuum. The optical density was measured by Pierce assay, and the absorbance at 474 nm was measured.

The above-described procedures were repeated on samples using different concentrations of CC160 clay as recited in Table 7. Table 7 recites the concentrations of activated carbon or clay and the absorbances at 474 nm before and after treatment and filtration.

TABLE 7

| | Activated | | | |
|---|---|---|---|---|
| | carbon | Clay | Clay | Clay |
| | (0.3%) | (0.3%) | (1%) | (5%) |
| Absorbance at | 0.297 | 0.297 | 0.297 | 0.297 |
| 474 nm before | | | | |
| treatment and | | | | |
| filtration | | | | |
| Absorbance at | 0.177 | 0.403 | 0.341 | 0.163 |
| 474 nm after | | | | |
| treatment and | | | | |
| filtration | | | | |

Without wishing to be bound by theory, these results may suggest that activated carbon is better at removing polyphenols than bentonite clay is.

### Example 12

This example investigated whether resin could be used in place of activated carbon.

Fresh supernatant (post-chitosan spindown in the extraction process) was obtained, and its pH was increased to 7.0 using NaOH. The starting optical density (“OD”) was measured by Pierce assay (Pierce™ 660 nm Protein Assay Reagent; Thermo Fisher Scientific, Waltham, Mass.), and the absorbance at 474 nm (Shimadzu PharmaSpec UV-1700; Shimadzu Scientific Instruments Incorporated, Columbia, Md.) was measured. To a 100 mL sample was added 0.3% activated carbon (Cabot Norit Americas Inc, Marshall, Tex.). The mixture was stirred for 1 minute, flowed through a 0.45 □ filter (Polyethersulfone (PES) Membrane Filters, 0.45 Micron; Sterlitech Corporation Inc, Kent, Wash.) followed by a 0.2 □ filter (Polyethersulfone (PES) Membrane Filters, 0.2 Micron; Sterlitech Corporation Inc, Kent, Wash.) on a Buchner funnel, collecting the filtrate in a flask. A sample was collected for optical density measurements. To further 100 mL samples, resin was added in the amount (% w/v) indicated in Table 8, and, for each sample, the mixture was stirred for the time indicated in Table 8 prior to pouring the mixture through the Buchner setup. Optical density measurements were taken on each sample.

TABLE 8

| MN200 | | | | | | |
|---|---|---|---|---|---|---|
| Purolite | 20%, | 5%, | 3%, | 10%, | 20%, | 5%, |
| resin | 2 min | 5 min | 10 min | 5 min | 10 min | 20 min |
| Supernat | 0.697 | 0.697 | 0.697 | 0.697 | 0.697 | 0.697 |
| OD | | | | | | |
| (Pierce) | | | | | | |
| “pre” | | | | | | |
| OD | 0.555 | 0.617 | 0.594 | 0.600 | 0.578 | 0.541 |
| (Pierce) | | | | | | |
| post | | | | | | |
| resin | | | | | | |
| binding/ | | | | | | |
| filter | | | | | | |
| Supernatant | 0.308 | 0.308 | 0.308 | 0.308 | 0.308 | 0.308 |
| OD | | | | | | |
| (abs at | | | | | | |
| 474 nm) | | | | | | |
| “pre” | | | | | | |
| OD | 0.137 | 0.168 | 0.157 | 0.137 | 0.102 | 0.124 |
| (474 nm) | | | | | | |
| post | | | | | | |
| resin | | | | | | |
| binding/ | | | | | | |
| filter | | | | | | |
| Pierce | 0.796269727403156 | 0.885222381635581 | 0.852223816355811 | 0.860832137733142 | 0.829268292682927 | 0.776183644189383 |
| post/pre | | | | | | |
| ratio | | | | | | |
| Orange | 0.444805194805195 | 0.545454545454546 | 0.50974025974026 | 0.444805194805195 | 0.331168831168831 | 0.402597402597403 |
| (474) | | | | | | |
| post/pre | | | | | | |
| ratio | | | | | | |

| MN200 | | |
|---|---|---|
| Purolite | 10%, | 10%, |
| resin | 2 min | 10 min |
| Supernat | 0.697 | 0.697 |
| OD | | |
| (Pierce) | | |
| “pre” | | |
| OD | 0.581 | 0.579 |
| (Pierce) | | |
| post | | |
| resin | | |
| binding/ | | |
| filter | | |
| Supernatant | 0.308 | 0.308 |
| OD | | |
| (abs at | | |
| 474 nm) | | |
| “pre” | | |
| OD | 0.142 | 0.112 |
| (474 nm) | | |
| post | | |
| resin | | |
| binding/ | | |
| filter | | |
| Pierce | 0.833572453371593 | 0.830703012912482 |
| post/pre | | |
| ratio | | |
| Orange | 0.461038961038961 | 0.363636363636364 |
| (474) | | |
| post/pre | | |
| ratio | | |

An aspect of the Example was to determine the binding time and concentration for which the orange OD is lowest while maintaining the highest possible Pierce assay reading, correlating to protein concentration. This can be seen graphically by the largest separation between the Pierce ratio line and the 474 ratio line. The experimental group with the largest separation was 20% for 10 minutes, with 83% of protein retained by Pierce assay and reduction of orange color to 33%.

### Example 13

This example investigated the effectiveness of a resin (Purolite MN200; Purolite Corporation, Kings of Prussia, Pa.) at removing colored compounds, as compared to activated carbon.

Fresh supernatant (post-chitosan spindown in the extraction process) was obtained, and the pH was increased to 7.0 with NaOH. The starting optical density (“OD”) was measured by Pierce assay (Pierce™ 660 nm Protein Assay Reagent; Thermo Fisher Scientific, Waltham, Mass.), and the absorbance at 474 nm (Shimadzu PharmaSpec UV-1700; Shimadzu Scientific Instruments Incorporated, Columbia, Md.) was measured. To a 100 mL sample was added 0.3% activated carbon (Cabot Norit Americas Inc, Marshall, Tex.). The mixture was stirred for 1 minute, flowed through a 0.45 □ filter (Polyethersulfone (PES) Membrane Filters, 0.45 Micron; Sterlitech Corporation Inc, Kent, Wash.) followed by a 0.2 □ filter (Polyethersulfone (PES) Membrane Filters, 0.2 Micron; Sterlitech Corporation Inc, Kent, Wash.) on a Buchner funnel, collecting the filtrate in a flask. A sample was collected for optical density measurements. To further 100 mL samples, resin was added in the amount (% w/v) indicated in Table 9, and, for each sample, the mixture was stirred for the time indicated in Table 9 prior to pouring the mixture through the Buchner setup. Optical density measurements (Pierce at 710 nm, Abs at 474 nm) were taken on each sample.

TABLE 9

| Material | Activated carbon (AC)-fine, Sigma | | | |
|---|---|---|---|---|
| Amount | | | | |
| and | | | | |
| residence | 0.3%, | 0.5%, | 1%, | 2%, |
| time | 1 min | 1 min | 1 min | 1 min |
| Supernatant | 0.619 | 0.619 | 0.619 | 0.619 |
| OD | | | | |
| (Pierce) | | | | |
| “pre” | | | | |
| OD | 0.553 | 0.518 | 0.521 | 0.344 |
| (Pierce) | | | | |
| post resin | | | | |
| binding/ | | | | |
| filter | | | | |
| Supernatant | 0.282 | 0.282 | 0.282 | 0.282 |
| OD | | | | |
| (abs at | | | | |
| 474 nm) | | | | |
| “pre” | | | | |
| OD | 0.243 | 0.170 | 0.107 | 0.068 |
| (474 nm) | | | | |
| post resin | | | | |
| binding/ | | | | |
| filter | | | | |
| Pierce | 0.893376413570275 | 0.836833602584814 | 0.841680129240711 | 0.555735056542811 |
| post/pre | | | | |
| ratio | | | | |
| Orange | 0.861702127659575 | 0.602836879432624 | 0.379432624113475 | 0.24113475177305 |
| (474) | | | | |
| post/pre | | | | |
| ratio | | | | |
| Material | Resin (MN200-Purolite) | | | |
| Amount | | | | |
| and | | | | |
| residence | 20%, | 5%, | 10%, | 10%, |
| time | 10 min | 20 min | 2 min | 10 min |
| Supernatant | 0.619 | 0.619 | 0.619 | 0.619 |
| OD | | | | |
| (Pierce) | | | | |
| “pre” | | | | |
| OD | 0.466 | 0.556 | 0.589 | 0.576 |
| (Pierce) | | | | |
| post resin | | | | |
| binding/ | | | | |
| filter | | | | |
| Supernatant | 0.282 | 0.282 | 0.282 | 0.282 |
| OD | | | | |
| (abs at | | | | |
| 474 nm) | | | | |
| “pre” | | | | |
| OD | 0.172 | 0.219 | 0.226 | 0.220 |
| (474 nm) | | | | |
| post resin | | | | |
| binding/ | | | | |
| filter | | | | |
| Pierce | 0.752827405492731 | 0.898222940226171 | 0.951534733441034 | 0.930533117932149 |
| post/pre | | | | |
| ratio | | | | |
| Orange | 0.609929078014184 | 0.776595744680851 | 0.801418439716312 | 0.780141843971631 |
| (474) | | | | |
| post/pre | | | | |
| ratio | | | | |

Without wishing to be bound by theory, it is believed that the large gap between the Pierce OD ratio and the orange OD ratio seen in the 1% activated carbon, 1 minute group may be an error, as this was not observed in the previous trial. Without wishing to be bound by theory, these results may suggest that activated carbon is more efficient than the tested resins in removing color but that activated carbon removes more nitrogenous compounds based on Pierce assay.

### Example 14

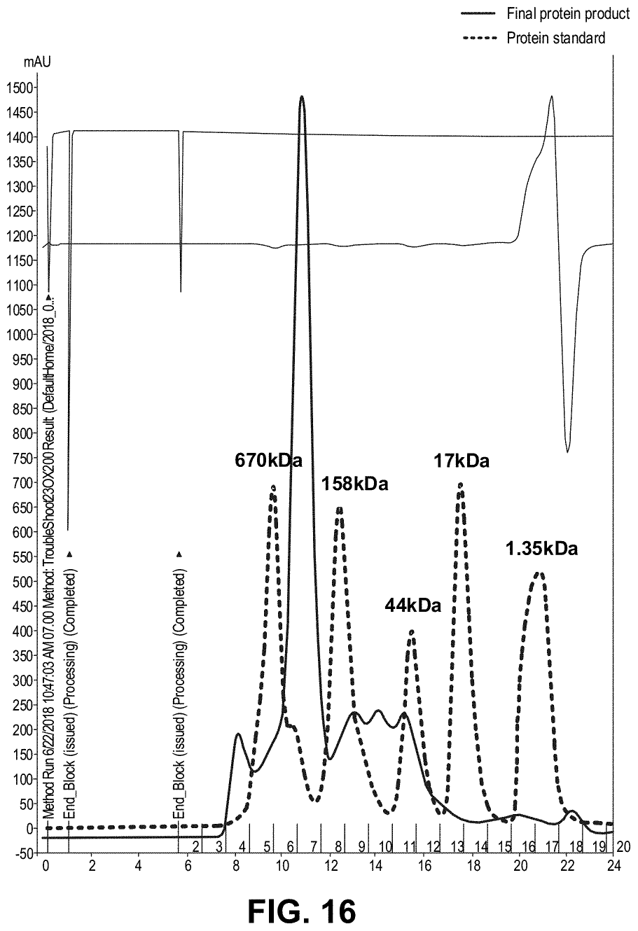

Final plant protein powder was diluted at a concentration of 10 mg/mL in deionized water. The sample was analyzed using fast protein liquid chromatography (FPLC) using a gel filtration column (Superdex 200; GE Healthcare Bio-Sciences Corp, Westborough, Mass.). A molecular weight standard (Bio-Rad Laboratories, INC, Hercules, Calif.) was subsequently run to approximate the molecular size of individual proteins and protein complexes in the sample of interest. FIG. 16 depicts a chromatogram of final protein product and protein standard. Chromatogram protein peak analysis indicated that the protein of interest (Rubisco) was eluted from the column at a molecular weight near to but less than 670 kDa as measured by the molecular weight protein standard.

### Example 15

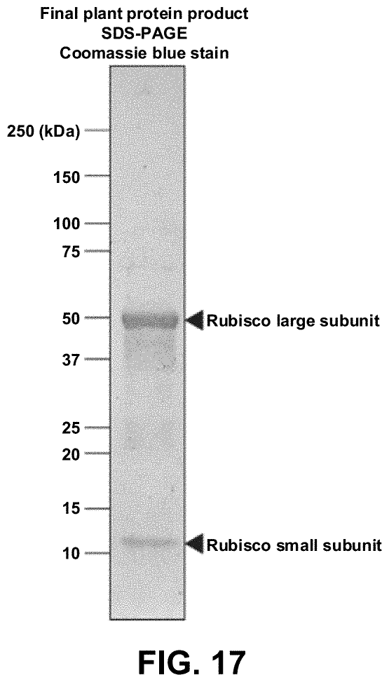

This example used SDS-PAGE electrophoresis to visualize Lemna plant protein purity and complexity via Coomassie staining. A small sample of plant protein extract of final purified protein product was analyzed on a 4-15% SDS-PAGE gel (Bio-Rad Laboratories, INC, Hercules, Calif.). Visualization of proteins was performed by utilizing Coomassie dye to stain the proteins blue in the gel. FIG. 17 depicts the SDS-PAGE gel. Without wishing to be bound by theory, it is believed that these results suggest that the final protein product consists primarily of Rubisco enzyme and that the individual large and small subunits of Rubisco can be readily detected by SDS-PAGE Coomassie staining under denatured and reducing conditions.

### Example 16

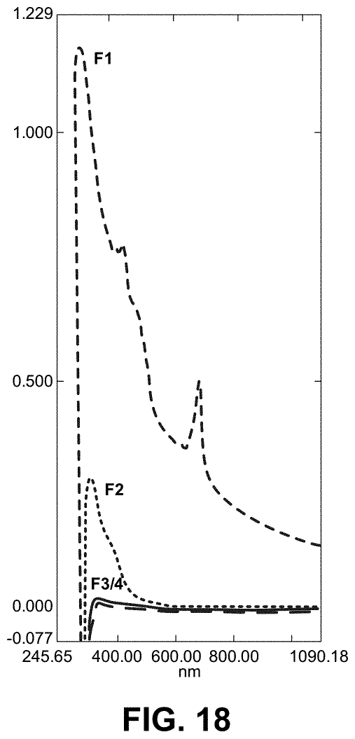

This example studied the removal of chlorophyll, polyphenols, and other light absorbing molecules as characterized and quantified by spectrophotometry. Samples from each step of the purification process were characterized by a spectrophotometer (Shimadzu PharmaSpec UV-1700; Shimadzu Scientific Instruments Incorporated, Columbia, Md.) through the purification process. Samples were scanned from 1100 nm down to 245 nm. FIG. 18 depicts an absorbance spectrum. The Fraction 1 peak (“F1”) corresponds to the signal detected from filtrate after the first liquid/solid separation step. The Fraction 4 (“F4”) peak corresponds to a sample taken after 0.2 □m microfiltration. Without wishing to be bound by theory, it is believed that the absorbance peaks detected from the scanning spectrophotometer indicated efficient removal of light absorbing molecules throughout the process.

We claim:

1. A composition, wherein the composition is a food product source in the form of a gel and comprises:
a protein isolate from Lemna plant biomass, wherein at least 60% of the protein isolate comprises ribulose-1,5-bisphosphate carboxylase/oxygenase (RuBisCO) protein; and
an aqueous solution.

2. The composition of claim 1, wherein at least 65% of the protein isolate comprises RuBisCO protein.

3. The composition of claim 1, wherein at least 70% of the protein isolate comprises RuBisCO protein.

4. The composition of claim 1, wherein at least 75% of the protein isolate comprises RuBisCO protein.

5. The composition of claim 1, wherein the RuBisCO protein comprises the RuBisCO protein large subunit.

6. The composition of claim 1, wherein the RuBisCO protein comprises the RuBisCO protein small subunit.

7. The composition of claim 1, wherein the protein isolate comprises amino acid chains that are not unfolded.

8. The composition of claim 1, wherein the Lemna plant of the Lemna plant biomass comprises Lemna minor.

9. The composition of claim 1, wherein the Lemna plant of the Lemna plant biomass is Lemna minor.

10. The composition of claim 1, wherein the aqueous solution is water.

11. The composition of claim 1, wherein the protein isolate from Lemna plant biomass comprises a protein purity of at least 80% by dry weight.

12. The composition of claim 1, wherein the composition further comprises a salt.

13. The composition of claim 12, wherein the salt comprises potassium phosphate, calcium chloride, or sodium hydroxide.

14. The composition of claim 1, wherein the composition has a pH level from 6.8 to 7.3.

15. The composition of claim 1, wherein the composition comprises at least 2% w/v of the protein isolate.

16. The composition of claim 1, wherein the composition comprises 2% w/v of the protein isolate.
